(19) United States
(12) Patent Application Publication
(10) Pub. No.: US 2021/0060459 A1
(43) Pub. Date: Mar. 4, 2021

(54) FILTRATION DEVICE AND FILTRATION METHOD

(71) Applicant: Murata Manufacturing Co., Ltd., Nagaokakyo-shi (JP)

(72) Inventors: Takashi Kondo, Nagaokakyo-shi (JP); Satoshi Murata, Nagaokakyo-shi (JP); Toshikazu Kawaguchi, Nagaokakyo-shi (JP); Miwako Nishikawa, Nagaokakyo-shi (JP)

(21) Appl. No.: 17/097,307

(22) Filed: Nov. 13, 2020

Related U.S. Application Data
(63) Continuation of application No. PCT/JP2019/020622, filed on May 24, 2019.

(30) Foreign Application Priority Data
Aug. 23, 2018 (JP) 2018-155944
Oct. 18, 2018 (JP) 2018-196415
Jan. 11, 2019 (JP) 2019-003726

(51) Int. Cl. B01D 29/23

(52) U.S. Cl. CPC B01D 29/23; B01D 2201/309; B01D 2201/296; B01D 2201/287

(57) ABSTRACT

A filtration device that includes a cylindrical body and a filtration part. The cylindrical body has a first open end and a second closed end with an end wall. The filtration part is on a circumferential portion of the cylindrical body and has through-holes.

### CROSS REFERENCE TO RELATED APPLICATIONS

The present application is a continuation of International application No. PCT/JP2019/020622, filed May 24, 2019, which claims priority to Japanese Patent Application No. 2018-155944, filed Aug. 23, 2018, Japanese Patent Application No. 2018-196415, filed Oct. 18, 2018, and Japanese Patent Application No. 2019-003726, filed Jan. 11, 2019, the entire contents of each of which are incorporated herein by reference.

### FIELD OF THE INVENTION

The present invention relates to a filtration device and a filtration method.

### BACKGROUND OF THE INVENTION

Known devices used to filter liquid that contains filtration targets include a device disclosed in Japanese Unexamined Patent Application Publication No. 2013-210239 (Patent Document 1), or more specifically, a pretreatment device for on-line measurement. The device disclosed in Patent Document 1 is a pretreatment device for on-line measurement of the quality of water in a water system and includes filtration means incorporating an external pressure type hollow fiber membrane for cross-filter filtration.

### SUMMARY OF THE INVENTION

In recent years, there has been a demand that filtration be performed with efficiency.

It is an object of the present invention to provide a filtration device and a filtration method that enable efficient filtration.

A filtration device according to an aspect of the present invention includes: a cylindrical body having a first open end, a second closed end opposite the first open end, and a plurality of frame members that define openings between an inside and an outside of the cylindrical body; and a cylindrical filter having through holes, the cylindrical filter being attached to the plurality of frame members so as to wrap around a circumferential portion of the cylindrical body.

A filtration method according to another aspect of the present invention includes: setting up a filtration device including a cylindrical body, a filtration part, and a reservoir part, the cylindrical body a first open end, a second closed end, a plurality of frame members that define openings between an inside and an outside of the cylindrical body, the filtration part including a cylindrical filter having through holes attached to the plurality of frame members so as to wrap around a circumferential portion of the cylindrical body, the reservoir part being located at the second closed end of the cylindrical body and configured to store a filtration target and a liquid; introducing a liquid containing a filtration target into the filtration device; storing the filtration target and the liquid in the reservoir part; draining the liquid from the filtration part, with the filtration target being caught in the filtration part; and collecting the filtration target and the liquid from the reservoir part.

According to the present invention, a filtration device and a filtration method that enable efficient filtration are provided.

### BRIEF DESCRIPTION OF THE DRAWINGS

FIG. 1 is a schematic perspective view of an example of a filtration device according to Embodiment 1 of the present invention.

FIG. 2 is a schematic front view of the filtration device according to Embodiment 1 of the present invention.

FIG. 3 is a schematic sectional view of the filtration device according to Embodiment 1 of the present invention.

FIG. 4 is a schematic view of the filtration device according to Embodiment 1 of the present invention, with a filtration part being omitted.

FIG. 5 is an enlarged partial perspective view of a filtration part presented as an example.

FIG. 6 is a schematic partial view of the filtration part illustrated in FIG. 5 and seen in the thickness direction.

FIG. 7 is a view of the filtration device according to Embodiment 1 of the present invention, schematically illustrating the configuration of the filtration device in use.

FIG. 8 is a schematic sectional view of the filtration device according to Embodiment 1 of the present invention, schematically illustrating the filtration device in use.

FIG. 9 is a flowchart of an example of a filtration method according to Embodiment 1 of the present invention.

FIG. 10A illustrates a step that may be included in the filtration method according to Embodiment 1 of the present invention.

FIG. 10B illustrates another step that may be included in the filtration method according to Embodiment 1 of the present invention.

FIG. 10C illustrates still another step that may be included in the filtration method according to Embodiment 1 of the present invention.

FIG. 10D illustrates still another step that may be included in the filtration method according to Embodiment 1 of the present invention.

FIG. 10E illustrates still another step that may be included in the filtration method according to Embodiment 1 of the present invention.

FIG. 10F illustrates still another step that may be included in the filtration method according to Embodiment 1 of the present invention.

FIG. 11A schematically illustrates a filtration device according to a modification of Embodiment 1 of the present invention.

FIG. 11B schematically illustrates a filtration device according to another modification of Embodiment 1 of the present invention.

FIG. 12 schematically illustrates the configuration of a filtration device according to still another modification of Embodiment 1 of the present invention.

FIG. 13 is a schematic sectional view of the filtration device according to the modification of Embodiment 1 of the present invention.

FIG. 14 schematically illustrates a filtration device according to still another modification of Embodiment 1 of the present invention.

FIG. 15A schematically illustrates a filtration device according to still another modification of Embodiment 1 of the present invention.

FIG. 15B schematically illustrates a filtration device according to still another modification of Embodiment 1 of the present invention.

FIG. 15C schematically illustrates a filtration device according to still another modification of Embodiment 1 of the present invention.

FIG. 16A schematically illustrates a filtration device according to still another modification of Embodiment 1 of the present invention.

FIG. 16B is a schematic exploded view of the filtration device according to the modification of Embodiment 1 of the present invention.

FIG. 17 schematically illustrates a filtration device according to still another modification of Embodiment 1 of the present invention.

FIG. 18 is a flowchart of an example of a filtration method according to Embodiment 2 of the present invention.

FIG. 19A illustrates another step that may be included in the filtration method according to Embodiment 2 of the present invention.

FIG. 19B illustrates still another step that may be included in the filtration method according to Embodiment 2 of the present invention.

FIG. 19C illustrates still another step that may be included in the filtration method according to Embodiment 2 of the present invention.

FIG. 19D illustrates still another step that may be included in the filtration method according to Embodiment 2 of the present invention.

FIG. 19E illustrates still another step that may be included in the filtration method according to Embodiment 2 of the present invention.

FIG. 19F illustrates still another step that may be included in the filtration method according to Embodiment 2 of the present invention.

FIG. 19G illustrates still another step that may be included in the filtration method according to Embodiment 2 of the present invention.

FIG. 19H illustrates still another step that may be included in the filtration method according to Embodiment 2 of the present invention.

FIG. 19I illustrates still another step that may be included in the filtration method according to Embodiment 2 of the present invention.

FIG. 20 is a schematic perspective view of an example of a filtration system according to Embodiment 3 of the present invention.

FIG. 21 is a schematic front view of an example of a filtration system according to Embodiment 3 of the present invention.

FIG. 22 is a schematic sectional view of the filtration system taken along line A-A in FIG. 21.

FIG. 23A illustrates an action that may be performed by the filtration system according to Embodiment 3 of the present invention.

FIG. 23B illustrates another action that may be performed by the filtration system according to Embodiment 3 of the present invention.

FIG. 23C illustrates still another action that may be performed by the filtration system according to Embodiment 3 of the present invention.

FIG. 23D illustrates still another action that may be performed by the filtration system according to Embodiment 3 of the present invention.

FIG. 23E illustrates still another action that may be performed by the filtration system according to Embodiment 3 of the present invention.

FIG. 24 schematically illustrates a filtration system according to a modification of Embodiment 3 of the present invention.

FIG. 25 schematically illustrates a filtration system according to a modification of Embodiment 3 of the present invention.

FIG. 26A illustrates an action that may be performed by the filtration system according to the modification of Embodiment 3 of the present invention.

FIG. 26B illustrates another action that may be performed by the filtration system according to the modification of Embodiment 3 of the present invention.

FIG. 26C illustrates still another action that may be performed by the filtration system according to the modification of Embodiment 3 of the present invention.

FIG. 26D illustrates still another action that may be performed by the filtration system according to the modification of Embodiment 3 of the present invention.

FIG. 26E illustrates still another action that may be performed by the filtration system according to the modification of Embodiment 3 of the present invention.

FIG. 27 is a flowchart of an example of a filtration method according to Embodiment 4 of the present invention.

FIG. 28A illustrates a step that may be included in the filtration method according to Embodiment 4 of the present invention.

FIG. 28B illustrates another step that may be included in the filtration method according to Embodiment 4 of the present invention.

FIG. 28C illustrates still another step that may be included in the filtration method according to Embodiment 4 of the present invention.

FIG. 28D illustrates still another step that may be included in the filtration method according to Embodiment 4 of the present invention.

FIG. 29 is a schematic sectional view of an example of a filtration device according to Embodiment 5 of the present invention.

FIG. 30 is a flowchart of an example of a filtration method according to Embodiment 5 of the present invention.

FIG. 31A illustrates a step that may be included in the filtration method according to Embodiment 5 of the present invention.

FIG. 31B illustrates another step that may be included in the filtration method according to Embodiment 5 of the present invention.

FIG. 31C illustrates still another step that may be included in the filtration method according to Embodiment 5 of the present invention.

FIG. 31D illustrates still another step that may be included in the filtration method according to Embodiment 5 of the present invention.

FIG. 32 is a schematic sectional view of an example of a filtration device according to a modification of Embodiment 5 of the present invention.

FIG. 33 is a schematic sectional view of the filtration device according to the modification of Embodiment 5 of the present invention, illustrating an action that may be performed by the filtration device.

FIG. 34 is a schematic sectional view of an example of a filtration device according to Embodiment 6 of the present invention.

FIG. 35A illustrates an action that may be performed by the filtration device according to Embodiment 6 of the present invention.

FIG. 35B illustrates another action that may be performed by the filtration device according to Embodiment 6 of the present invention.

FIG. 36 is a schematic sectional view of a filtration device according to a modification of Embodiment 6 of the present invention.

### DETAILED DESCRIPTION OF THE PREFERRED EMBODIMENTS

Cells taken out as filtration targets by filtration carried out by a filtration device may be exposed to the atmosphere while being collected, and as a result, the cells become less active. This is the reason why cells having undergone filtration need to be immersed in liquid when being collected.

A filtration device for cross-flow filtration includes mainly a pump, pipes, a filtration part, and a receptacle, which constitute a circulation path. Liquid containing filtration targets and stored in a receptacle is pumped into the pipes by the pump. The liquid pumped into the pipes undergoes cross-flow filtration while passing through a portion to which the filtration part is provided. Cross-flow filtration is a process in which part of the liquid flowing through the pipe is drained out of the pipe from the filtration part, and the residual liquid in the pipe returns to the receptacle.

The residual filtration targets and the residual liquid in the circulation channel (e.g., pipes) may be left upon cross-flow filtration. It is difficult to collect the residual filtration targets in the circulation channel. The volume of liquid that may be collected with filtration targets cannot be controlled. Furthermore, cross-flow filtration necessitates the use of a device having complex configuration.

A study regarding a filtration device and a filtration method that enable efficient filtration has been conducted by the inventors in the present application to address these problems and has resulted in the present invention, which will be described below.

A filtration device according to an aspect of the present invention includes: a cylindrical body having a first open end and a second closed end opposite the first open end; and a filtration part on a circumferential portion of the cylindrical body and having through-holes. This enables efficient filtration.

The filtration part of the filtration device may extend all around the circumferential portion of the cylindrical body. This is conducive to a short-time filtration.

The filtration part of the filtration device may extend halfway or less around the circumferential portion of the cylindrical body. The makes the filtration position easily changeable and accordingly enables efficient filtration.

The filtration device may also include a reservoir part provided to the second end of the cylindrical body and located below the filtration part, with the first end of the cylindrical body being located at a level above the second end. This provides ease of collecting the filtration target.

When cross sections of the reservoir part of the filtration device are taken in directions orthogonal to a direction of connection between the first open end and the second closed end of the cylindrical body, an opening cross-sectional area of a portion of the reservoir part close to the second closed end of the cylindrical body may be smaller than an opening cross-sectional area of a portion of the reservoir part close to the filtration part. This helps store the filtration target and the liquid in the reservoir part and provides greater ease of collecting the filtration target.

The reservoir part of the filtration device has an inner wall, which may include an inclined portion inclined toward the second closed end of the cylindrical body. This helps store the filtration target and the liquid in the reservoir part and provides greater ease of collecting the filtration target.

The inclined portion may be inclined toward the center of the cylindrical body of the filtration device. This provides greater ease of collecting the filtration target.

The reservoir part of the filtration device has an outer wall, which may include a protruding portion protruding toward the second closed end of the cylindrical body. The liquid drained out of the cylindrical body from the filtration part flows along the outer wall of the reservoir part accordingly. This eliminates or reduces the possibility that the liquid drained out of the cylindrical body from the filtration part will splatter.

The protruding portion has a side face, which may be inclined toward the center of the cylindrical body of the filtration device. This eliminates or further reduces the possibility that the liquid drained out of the cylindrical body from the filtration part will splatter.

The cylindrical body of the filtration device may include frame members that define openings, each of which is an interface between the inside and the outside of the cylindrical body. The filtration part of the filtration device may be a cylindrical filter and may be attached to the frame members. The filtration part is easily provided on the circumferential portion of the cylindrical body accordingly.

The filtration device may also include a liquid-retaining receptacle close to the second closed end of the cylindrical body. The liquid drained out of the cylindrical body from the filtration part may be received in the liquid-retaining receptacle accordingly.

The cylindrical body of the filtration device may be made of a resin through which the inside of the cylindrical body is visible from outside the cylindrical body. This enables viewing of the filtration target and the liquid that are stored in the reservoir part.

The filtration part of the filtration device may be a filter made mainly of a metal and/or a metal oxide. This is conducive to a short-time filtration.

A filtration method according to another aspect of the present invention includes: setting up a filtration device including a cylindrical body, a filtration part, and a reservoir part, the cylindrical body having a first open end and a second closed end opposite the open end, the filtration part being provided on a circumferential portion of the cylindrical body and having through-holes, the reservoir part being provided to the second closed end of the cylindrical body and located below the filtration part, the reservoir part being configured to store a filtration target and a liquid therein; introducing a liquid containing a filtration target into the filtration device; storing the filtration target and the liquid in the reservoir part; draining the liquid from the filtration part, with the filtration target being caught in the filtration part; and collecting the filtration target and the liquid that are stored in the reservoir part. This enables efficient filtration.

The filtration device used in the filtration method may also include a liquid-retaining receptacle close to the second closed end of the cylindrical body, and the step of draining the liquid from the filtration part includes retaining, in the liquid-retaining receptacle, the liquid drained from the filtration part. The liquid drained out of the cylindrical body from the filtration part may be received in the liquid-retaining receptacle accordingly.

Embodiment 1 of the present invention will be described below with reference to the accompanying drawings. Components in the drawings are exaggerated for easy-to-understand illustration.

### Embodiment 1

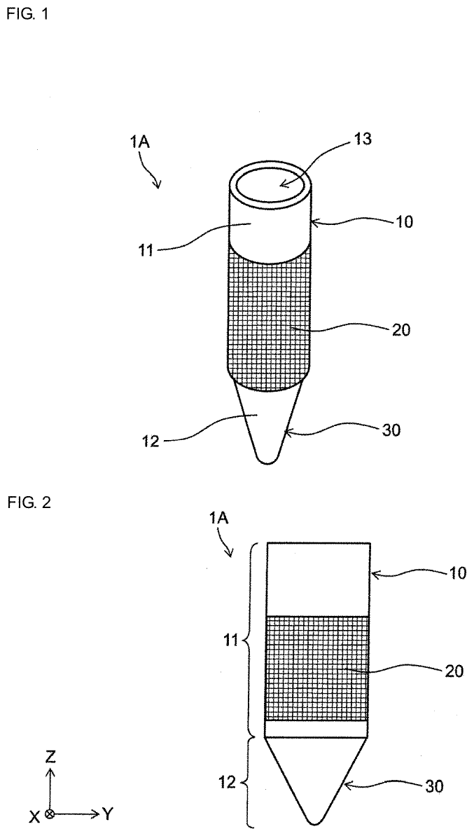

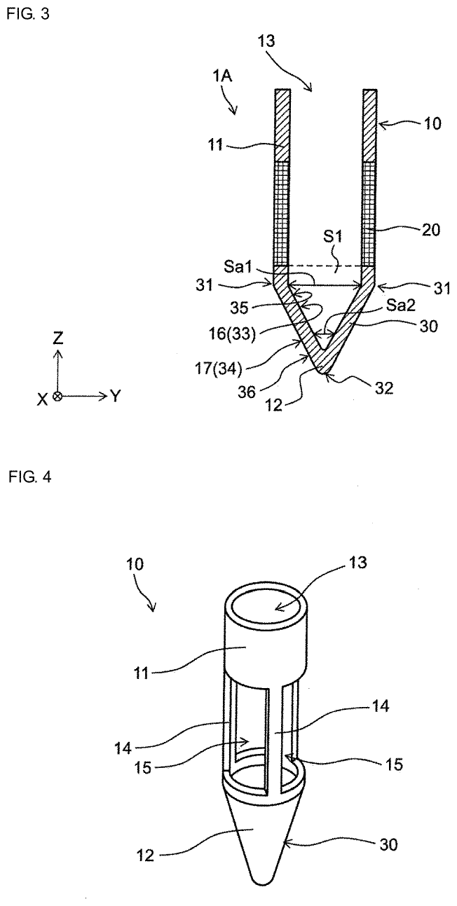

FIG. 1 is a schematic perspective view of an example of a filtration device 1A according to Embodiment 1 of the present invention. FIG. 2 is a schematic front view of the filtration device 1A according to Embodiment 1 of the present invention. FIG. 3 is a schematic sectional view of the filtration device 1A according to Embodiment 1 of the present invention. The lateral direction, the longitudinal direction, and the height direction of the filtration device 1A are denoted by X, Y, and Z, respectively.

As illustrated in FIGS. 1 to 3, the filtration device 1A includes a cylindrical body 10 and a filtration part 20. The cylindrical body 10 has two ends. The filtration part 20 is provided on a circumferential portion 11 of the cylindrical body 10 and has through-holes.

A first end of the cylindrical body 10 of the filtration device 1A is located at a level above a second end. For example, the cylindrical body 10 extends in the vertical direction (i.e., in the Z direction), with the first end of the cylindrical body 10 being located at an elevation higher than the second end. The first end of the cylindrical body 10 has an opening 13. The second end of the cylindrical body 10 is closed with an end wall 12. The end wall 12 with which the second end of the cylindrical body 10 is closed defines a reservoir part 30, which is located below the filtration part 20. Filtration targets and liquid may be stored in the reservoir part 30.

That is, the filtration device 1A according to Embodiment 1 includes the cylindrical body 10 with a bottom, the filtration part 20, and the reservoir part 30. The cylindrical body 10 includes the circumferential portion 11 and the end wall 12 with which the lower end (i.e., the second end) of the circumferential portion 11 is closed. The filtration part 20 is provided on the circumferential portion 11 of the cylindrical body 10 and has through-holes. The reservoir part 30 is provided to the second end of the cylindrical body 10 and located below the filtration part 20. Filtration targets and liquid may be stored in the reservoir part 30.

<Cylindrical Body>

The cylindrical body 10 has two ends. The cylindrical body 10 has the opening 13 at the first end and the end wall 12 at the second end. The cylindrical body 10 in Embodiment 1 is a receptacle having a bottom and the opening 13 at an upper portion of the cylindrical body 10. The cylindrical body 10 in Embodiment 1 has a cylindrical shape. The cylindrical body 10 includes the circumferential portion 11 and the end wall 12 with which the lower end (i.e., the second end) of the circumferential portion 11 is closed. The filtration part 20 having through-holes are provided on the circumferential portion 11 of the cylindrical body 10.

The cylindrical body 10 in Embodiment 1 extends in the vertical direction (i.e., the Z direction). That is, the circumferential portion 11 is a side wall of the cylindrical body 10, and the end wall 12 is a bottom portion of the cylindrical body 10.

The opening 13 is an inlet into which liquid containing filtration targets flows. The opening 13 is also an outlet through which liquid containing filtration targets flows out. That is, the opening 13 of the filtration device 1A is an inlet through which liquid containing filtration targets is drawn in.

FIG. 4 is a schematic view of the filtration device 1A according to Embodiment 1 of the present invention, with the filtration part 20 being omitted. As illustrated in FIG. 4, the circumferential portion 11 of the cylindrical body 10 includes frame members 14, which define openings 15. Each of the openings 15 is an interface between the inside and the outside of the cylindrical body 10. Specifically, the frame members 14 extend about halfway through the circumferential portion 11 of the cylindrical body 10 in the height direction of the cylindrical body 10 (i.e., in the Z direction). The frame members 14 are rod-like and spaced apart from each other. The openings 15 are each defined between two corresponding adjacent frame members 14.

In Embodiment 1, three frame members 14 extend about halfway through the circumferential portion 11 of the cylindrical body 10 and are spaced uniformly. Three openings 15 are defined by the three frame members 14 spaced apart from each other. The area of the openings 15 viewed laterally is greater than the area of outer surfaces of the frame members 14.

As illustrated in FIGS. 1 to 3, the end wall 12 of the cylindrical body 10 defines the reservoir part 30 in which filtration targets and liquid may be stored. As illustrated in FIG. 3, the reservoir part 30 has an inner wall 33, which is formed by denting an inner surface 16 of the end wall 12 in the height direction of the cylindrical body 10 (i.e., in the Z direction). Specifically, the inner wall 33 of the reservoir part 30 is formed by recessing the inner surface 16 of the end wall 12 of the cylindrical body 10 downward in the vertical direction.

The reservoir part 30 is located below the filtration part 20. The reservoir part 30 in Embodiment 1 is defined by part of the circumferential portion 11 and the end wall 12, that is, by portions of the cylindrical body 10 that are located below the filtration part 20. In other words, the reservoir part 30 is defined by a lower portion of the cylindrical body 10, that is, by the portion below the lowermost end of the filtration part 20.

When cross sections of the reservoir part 30 are taken in directions (i.e., the X and Y directions) orthogonal to the direction of connection between the first end and the second end of the cylindrical body 10 (i.e., orthogonal to the Z direction), an opening cross-sectional area Sa2, which is a cross-sectional area of a portion of the reservoir part 30 close to the second end of the cylindrical body 10, is smaller than an opening cross-sectional area Sa1, which is a cross-sectional area of a portion of the reservoir part 30 close to the filtration part 20. That is, when cross sections of the reservoir part 30 are taken in directions (i.e., the X and Y directions) orthogonal to the height direction of the cylindrical body 10 (i.e., orthogonal to the Z direction), the opening cross-sectional area Sa2 of the lower portion of the reservoir part 30 is smaller than the opening cross-sectional area Sa1 of the upper portion of the reservoir part 30. The lower portion of the reservoir part 30 is close to a bottom portion (i.e., a lowermost end portion 32) of the reservoir part 30, and the upper portion of the reservoir part 30 is an opening of the reservoir part 30. When cross sections of the reservoir part 30 in Embodiment 1 are taken in directions (i.e., the X and Y directions) orthogonal to the height direction of the cylindrical body 10 (i.e., orthogonal to the Z direction), the opening cross-sectional area of the reservoir part 30 decreases with increasing proximity to the second end of the cylindrical body 10, that is, to the lower portion of the reservoir part 30. The decrease in the opening cross-sectional area of the reservoir part 30 may be stepwise or constant in a direction toward the second end of the cylindrical body 10, that is, in a downward direction.

Specifically, the reservoir part 30 includes a connection portion 31 and the lowermost end portion 32. The connection portion 31 forms connection between the circumferential portion 11 and the end wall 12 of the cylindrical body 10. The lowermost end portion 32 is located at a level below the connection portion 31. The lowermost end portion 32 is the lowermost portion of the reservoir part 30.

When cross sections of the reservoir part 30 are taken in the directions (i.e., the X and Y directions) orthogonal to the height direction of the cylindrical body 10 (i.e., orthogonal to the Z direction), the opening cross-sectional area of the reservoir part 30 decreases with increasing distance from the connection portion 31 and with increasing proximity to the lowermost end portion 32.

The inner wall 33 of the reservoir part 30 in Embodiment 1 includes an inclined portion 35, which is inclined toward the second end of the cylindrical body 10; that is, the inclined portion 35 is a downward slope. The inclined portion 35 is also inclined toward the center of the cylindrical body 10. Specifically, the inner wall 33 of the reservoir part 30 is dented so as to form a conical shape.

Filtration targets and liquid are stored in a space 51 within the reservoir part 30. The size of the space 51 is determined by the desired volume of liquid that is to be collected subsequent to filtration. That is, the size of the space 51 is determined according to the volume of liquid that is to be collected.

The reservoir part 30 has an outer wall 34, which is formed by thrusting an outer surface 17 of the end wall 12 of the cylindrical body 10 in the height direction of the cylindrical body 10 (i.e., in the Z direction). Specifically, the outer wall 34 of the reservoir part 30 protrudes downward in the vertical direction.

When the filtration device 1A is viewed laterally, the outer wall 34 of the reservoir part 30 tapers toward the second end of the cylindrical body 10, that is, tapers downward. Specifically, the outer wall 34 of the reservoir part 30 extends from the connection portion 31 and tapers down toward the lowermost end portion 32.

The outer wall 34 of the reservoir part 30 in Embodiment 1 includes a protruding portion 36, which protrudes toward the second end of the cylindrical body 10, that is, protrudes downward. The protruding portion 36 is has a side face inclined toward the center of the cylindrical body 10. Specifically, the outer wall 34 of the reservoir part 30 protrudes so as to form a conical shape.

The inner wall 33 and the outer wall 34 of the reservoir part 30 in Embodiment 1 are geometrically similar. Specifically, both the outer shape and the inner shape of the reservoir part 30 are conical. The conical outer shape and the conical inner shape of the reservoir part 30 each have a rounded tip.

The cylindrical body 10 is made of a resin through which the inside of the cylindrical body 10 is visible from outside the cylindrical body 10. The cylindrical body 10 is made of, for example, polypropylene, polyethylene terephthalate, polyethylene, polystyrene, or polyether ether ketone (PEEK).

<Filtration Part>

The filtration part 20 is a filter provided on the circumferential portion 11 of the cylindrical body 10 and has through-holes. Liquid containing filtration targets is filtered through the filtration part 20. Specifically, the filtration part 20 allows the liquid to pass therethrough, with the filtration targets caught in the filtration part 20.

The term “filtration target” herein refers a target that is contained in liquid and is to be taken out by filtration. The filtration target contained in the liquid may be a substance derived from living organisms such as cells (eukaryotes), bacteria (eubacteria), and viruses. Examples of cells (eukaryotes) include induced pluripotent stem cells (iPSCs), ES cells, stem cells, mesenchymal stem cells, mononuclear cells, single cells, cell clusters, floating cells, adherent cells, nerve cells, leukocytes, cells for regenerative medicine, autologous cells, cancer cells, circulating tumor cells (CTCs), HL-60 cells, HeLa cells, and fungi. Examples of bacteria (eubacteria) includes Escherichia coli and Mycobacterium tuberculosis.

Embodiment 1 describes a specific example in which the liquid is a cell suspension and the filtration targets are cells.

The filtration part 20 in Embodiment 1 is a filter having a cylindrical shape. The filtration part 20 is attached to the frame members 14 extending about halfway through the circumferential portion 11 of the cylindrical body 10. The filter is, for example, a rectangular, plate-like structure having a first principal surface and a second principal surface opposite to the first principal surface and is mounted on the frame members 14 in a manner so as to be wrapped around the circumferential portion 11 of the cylindrical body 10. The filtration part 20 surrounds the circumferential portion 11 of the cylindrical body 10 accordingly. That is, the filtration part 20 extends all around the circumferential portion 11 of the cylindrical body 10.

The filter provided as the filtration part 20 is made of metal. Specifically, the filter provided as the filtration part 20 is made mainly of a metal and/or a metal oxide. Constituents of the filtration part 20 may be gold, silver, copper, platinum, nickel, palladium, titanium, alloy of these metals, and oxides of these metals.

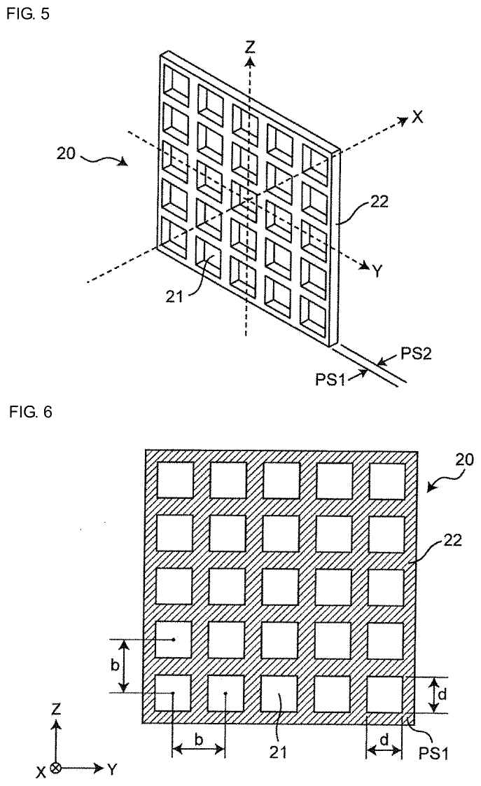

FIG. 5 is an enlarged partial perspective view of the filtration part 20 presented as an example. FIG. 6 is a schematic partial view of the filtration part 20 illustrated in FIG. 5 and seen in the thickness direction.

As illustrated in FIGS. 5 and 6, the filtration part 20 is a filter that is a plate-like structure having a first principal surface PS1 and a second principal surface PS2, which is opposite to the first principal surface PS1. The filter that is a plate-like structure is rolled up into a cylindrical shape to form the filtration part 20 in Embodiment 1. The first principal surface PS1 is a surface on the outside of the cylindrical filtration part 20, and the second principal surface PS2 is a surface on the inside of the cylindrical filtration part 20.

The filtration part 20 has through-holes 21, which extend through the first principal surface PS1 and the second principal surface PS2. Specifically, the through-holes 21 are provided in a filter substrate 22 of the filtration part 20.

The through-holes 21 are arranged in a periodic array on the first principal surface PS1 and the second principal surface PS2 of the filtration part 20. Specifically, the through-holes 21 of the filtration part 20 are provided at regular intervals and arranged in a matrix.

The through-holes 21 in Embodiment 1 have a square shape when viewed from the first principal surface PS1 of the filtration part 20, that is, when viewed in the X direction of the filtration device 1A. The shape of each through-hole 21 viewed in the X direction is not limited to a square and may be, for example, a rectangle, a circle, or an ellipse.

The through-holes 21 in Embodiment 1 are provided at regular intervals in two array directions parallel to the respective sides of the square viewed from the first principal surface PS1 of the filtration part 20 (i.e., viewed in the X direction), that is, are arranged at regular intervals in the Y and Z directions in FIG. 6. Owing to the through-holes 21 arranged in a square grid array, the filtration part 20 achieves a high aperture ratio, and the resistance to the flow of liquid through the filtration part 20 may be reduced accordingly. This enables a shortening of filtration time, and consequently, the stress on the filtration targets (cells) may be lightened.

Instead of being arranged in a square grid array, the through-holes 21 may be arranged in a quasi-periodic array or in a periodic array. The periodic array may be any quadrate array, examples of which include a rectangular array with intervals in one array direction not coinciding with intervals in the other array direction. Alternatively, the through-holes 21 may be arranged in a triangular grid array or in a regular triangle grid array. It is required that the filtration part 20 have more than one through-hole 21. The arrangement of the through-holes 21 is not limited to a particular pattern.

The intervals between the through-holes 21 are determined as appropriate according to the type (i.e., size, form, properties, or elasticity) or the volume of the filtration targets, namely, cells. As illustrated in FIG. 6, the intervals between the through-holes 21 are each denoted by b, which is a center-to-center distance of adjacent ones of the through-holes 21 viewed from the first principal surface PS1 of the filtration part 20. When the structure includes a periodic array of through-holes 21, the interval b between the through-holes 21 is, for example, more than the length of each side d of each through-hole 21 and not more than 10 times the length of each side d, and is more preferably not more than three times the length of each side d of each through-hole 21. The aperture ratio of the filtration part 20 may be, for example, not less than 10% and is preferably not less than 25%. The resistance to the flow of liquid through the filtration part 20 may thus be reduced. This enables a shortening of filtration time, and consequently, the stress on the filtration targets, namely, cells may be lightened. Dividing the area of the through-holes 21 by the projected area of a hypothetical example of the first principal surface PS1 with no through-hole 21 gives the aperture ratio.

The thickness of the filtration part 20 is preferably more than 0.1 times and not more than 100 times the dimension (the length of each side d) of each through-hole 21. The thickness of the filtration part 20 is more preferably more than 0.5 times and not more than 10 times the dimension (each side d) of each through-hole 21. The resistance imparted by the filtration part 20 to the flow of liquid may thus be reduced, and a shortening of filtration time may be achieved accordingly. Consequently, the stress on the filtration targets may be lightened.

It is preferred that the surface of the filtration part 20 that may come into contact with the liquid containing the filtration targets have a small surface roughness. The term “surface roughness” herein refers to the mean value of the difference between the maximum value and the minimum value as determined by a stylus profilometer at freely selected five spots on the surface that may come into contact with the liquid containing the filtration targets. In Embodiment 1, the surface roughness is preferably smaller than the size of each filtration target and is more preferably smaller than half the size of the filtration target. Specifically, the through-holes 21 on the second principal surface PS2 of the filtration part 20 are apertures in the same plane (i.e., in a YZ-plane). The filter substrate 22, which is the filtration part 20 except for the through-holes 21, is continuous and is formed as one member. This enables a reduction in the volume of filtration targets that get deposited on the second principal surface PS2 of the filtration part 20, and the resistance to the flow of liquid may be reduced accordingly.

In Embodiment 1, the liquid containing the filtration targets flows in the direction from the second principal surface PS2 on the inside of the filtration part 20 toward the first principal surface PS1 on the outside of the filtration part 20. It is thus preferred that the second principal surface PS2 have a small surface roughness.

Each through-hole 21 has a continuous wall surface that connects an aperture in the first principal surface PS1 and an aperture in the second principal surface PS2 to each other. Specifically, each through-hole 21 is provided in such a manner that its aperture in the first principal surface PS1 is projectable on its aperture in the second principal surface PS2. That is, each through-hole 21 is provided in such a manner that its aperture in the first principal surface PS1 overlaps its aperture in the second principal surface PS2 when the filtration part 20 is viewed from the first principal surface PS1.

The filtration part 20 in Embodiment 1 is a cylindrical filter having a diameter of 12 mm, a height of 22 mm, and a thickness of 2 μm. Each side d of each through-hole 21 having a square shape is 6 μm in length. The dimensions of the filtration part 20 are not limited to these values; that is, changes in dimensions are possible.

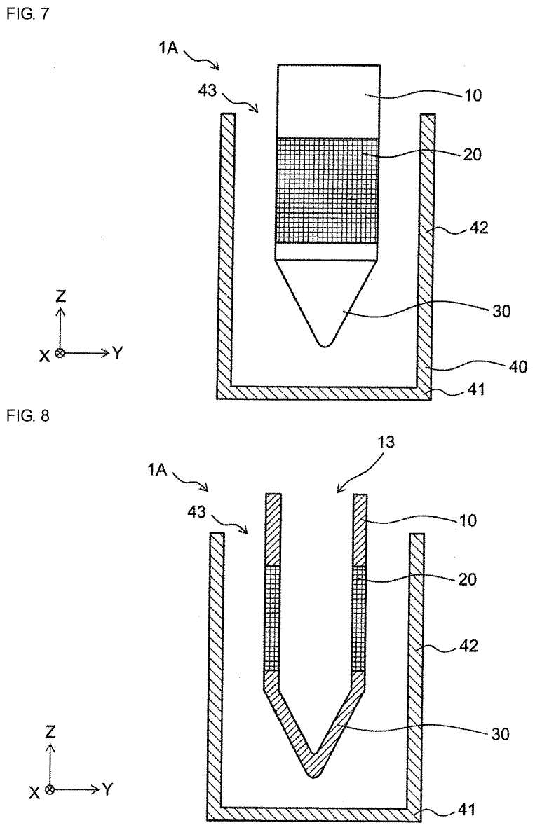

FIG. 7 is a view of the filtration device 1A according to Embodiment 1 of the present invention, schematically illustrating the configuration of the filtration device 1A in use. FIG. 8 is a schematic sectional view of the filtration device 1A according to Embodiment 1 of the present invention, illustrating the filtration device 1A in use. As illustrated in FIGS. 7 and 8, the filtration device 1A may include a liquid-retaining receptacle 40, in which liquid passing through the filtration part 20 and flowing on the outer side the cylindrical body 10 is received.

<Liquid-Retaining Receptacle>

The liquid-retaining receptacle 40 is close to the second end of the cylindrical body 10; that is, the liquid-retaining receptacle 40 is disposed below the cylindrical body 10. The liquid-retaining receptacle 40 has a bottom. Specifically, the liquid-retaining receptacle 40 has a bottom portion 41 and a side wall 42, which extends upward from an outer edge of the bottom portion 41. The liquid-retaining receptacle 40 has an opening 43, which is at an upper portion of the liquid-retaining receptacle 40. The liquid-retaining receptacle 40 in Embodiment 1 has a cylindrical shape. The inside diameter of the liquid-retaining receptacle 40 is greater than the outside diameter of the cylindrical body 10.

The cylindrical body 10 is inserted into the liquid-retaining receptacle 40 through the opening 43 of the liquid-retaining receptacle 40. The cylindrical body 10 may have a flange extending in the radial direction of the cylindrical body 10. With such a flange placed on an upper end of the liquid-retaining receptacle 40, the cylindrical body 10 is held in the liquid-retaining receptacle 40.

The liquid-retaining receptacle 40 may be a centrifuge tube.

[Filtration Method]

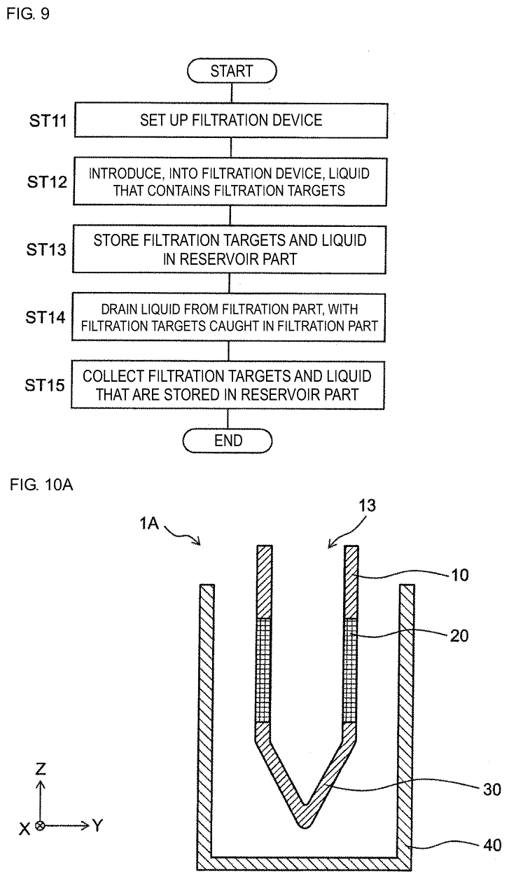

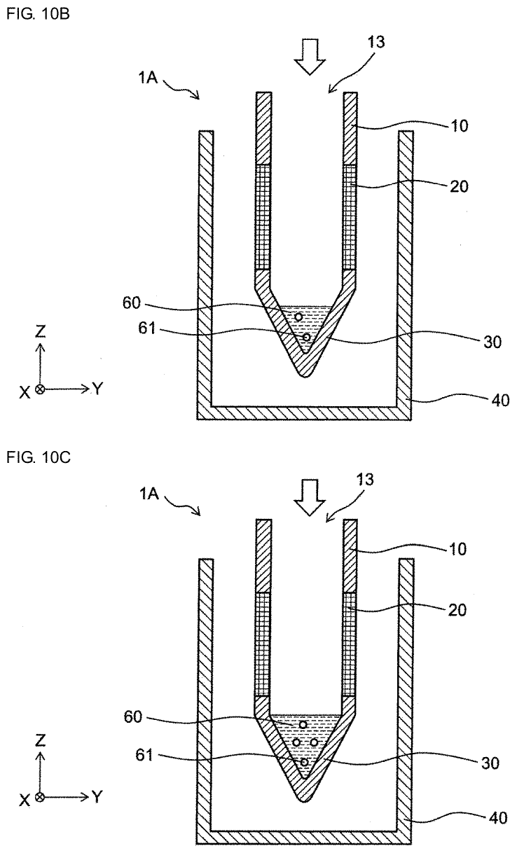

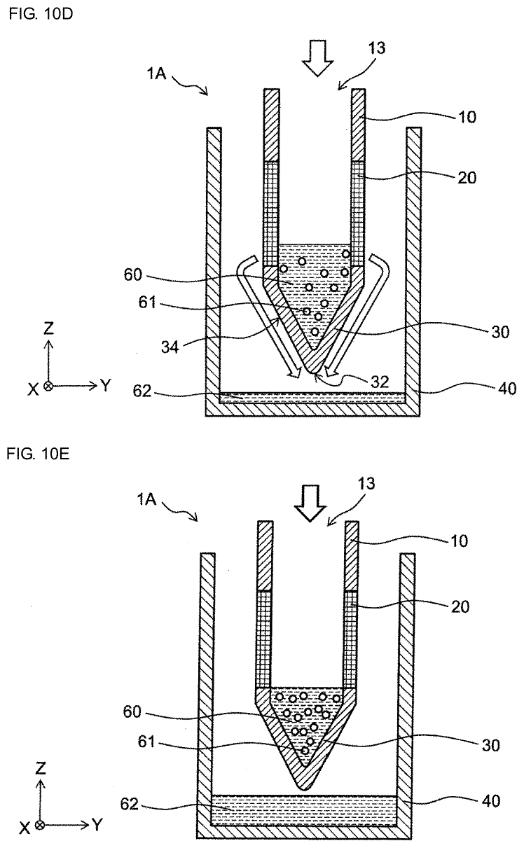

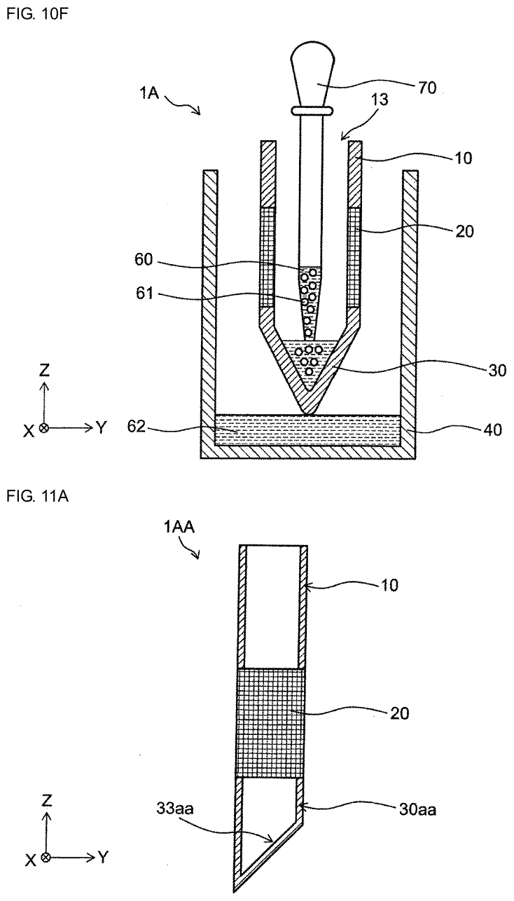

An example of a filtration method will be described below with reference to FIGS. 9 and 10A to 10F. FIG. 9 is a flowchart of an example of a filtration method according to Embodiment 1 of the present invention. FIGS. 10A to 10F illustrate steps that may be included in the filtration method according to Embodiment 1 of the present invention.

Referring to FIG. 9 and FIG. 10A illustrating Step ST11, the filtration device 1A is set up. Specifically, the cylindrical body 10 is disposed in the liquid-retaining receptacle 40.

Referring to FIG. 9 and FIG. 10B illustrating Step ST12, a liquid 60 and filtration targets 61, which are contained in the liquid 60, are introduced into the filtration device 1A. Specifically, the liquid 60 containing the filtration targets 61 is introduced into the cylindrical body 10 through the opening 13 of the cylindrical body 10.

Referring to FIG. 9 and FIG. 10C illustrating Step ST13, the filtration targets 61 and the liquid 60 are stored in the reservoir part 30 of the cylindrical body 10.

Referring to FIG. 9 and FIG. 10D illustrating Step ST14, the liquid 60 is drained from the filtration part 20, with the filtration targets 61 caught in the filtration part 20. The liquid 60 containing the filtration targets 61 is filtered accordingly. Specifically, the liquid 60 containing the filtration targets 61 is continuously introduced into the cylindrical body 10 through the opening 13 of the cylindrical body 10. When the liquid 60 containing the filtration targets 61 overflows from the reservoir part 30, the filtration targets 61 are caught in the filtration part 20 and remain in the cylindrical body 10. The liquid 60 overflowing from the reservoir part 30 passes through the filtration part 20 and is drained out of the cylindrical body 10.

Among the filtration targets 61 stored in the reservoir part 30, those that are larger than each through-hole 21 of the filtration part 20 cannot pass through the through-holes 21 of the filtration part 20 and are thus caught in the filtration part 20. Among the filtration targets 61 stored in the reservoir part 30, those that are smaller than each through-hole 21 of the filtration part 20 pass through the through-holes 21 of the filtration part 20 and are drained out of the cylindrical body 10.

Step ST14 in Embodiment 1 includes retaining, in the liquid-retaining receptacle 40, a liquid 62, which is the liquid passing through the filtration part 20 and drained out of the cylindrical body 10.

The liquid 60 drained from the filtration part 20 flows along the outer wall of the cylindrical body 10. Specifically, the liquid 60 flows toward the second end, that is, flows downward along the outer wall 34 of the reservoir part 30 of the cylindrical body 10. Owing to the conical shape formed by the outer wall 34 of the reservoir part 30, the liquid 60 flows toward the lowermost end portion 32. The liquid at the lowermost end portion 32 drips down onto a bottom portion of the liquid-retaining receptacle 40. In this way, the liquid 62 is collected in the liquid-retaining receptacle 40. This eliminates or reduces the possibility that the liquid 60 drained out of the cylindrical body 10 will splatter in the liquid-retaining receptacle 40.

Referring to FIG. 10E, filtration ends with the filtration targets 61 and the liquid 60 being stored in the reservoir part 30. Specifically, filtration ends with the space 51 in the reservoir part 30 being filled with the filtration targets 61 and the liquid 60.

Referring to FIG. 9 and FIG. 10F illustrating Step ST15, the filtration targets 61 and the liquid 60 that are stored in the reservoir part 30 are collected. Specifically, the filtration targets 61 and the liquid 60 that are stored in the reservoir part 30 are collected by using a collection tool 70. The collection tool 70 may be a pipette or a syringe.

The capacity in the space 51 of the reservoir part 30 is equal to the desired volume of the liquid 60 that is to be collected. The wording “is equal to” implies that a 10% tolerance is permitted. The use of the collection tool 70 in collecting the filtration targets 61 and the liquid 60 that are stored in the reservoir part 30 enables the collection of any desired volume of the liquid 60 containing the filtration targets 61.

The filtration device 1A and the filtration method according to Embodiment 1 produce the following effects.

The filtration device 1A includes the cylindrical body 10 and the filtration part 20. The cylindrical body 10 has two ends. The cylindrical body 10 has the opening 13 at first end and the end wall 12 at the second end. The filtration part 20 is provided on the circumferential portion 11 of the cylindrical body 10 and has the through-holes 21. The first end of the cylindrical body 10 of the filtration device 1A is located at a level above the second end. Specifically, the filtration device 1A includes the cylindrical body 10, the filtration part 20, and the reservoir part 30. The cylindrical body 10 has a bottom. The filtration part 20 is provided on the circumferential portion 11 of the cylindrical body 10 and has the through-holes 21. The reservoir part 30 is provided to the second end of the cylindrical body 10 and located below the filtration part 20. The filtration targets 61 and the liquid 60 may be stored in the reservoir part 30. This enables efficient filtration.

The liquid 60 containing the filtration targets 61 is introduced into the cylindrical body 10 through the opening 13 located at the upper portion of the cylindrical body 10. The filtration targets 61 and the liquid 60 are then stored in the reservoir part 30 adjoining the end wall 12 of the cylindrical body 10. Subsequent to filtration, the filtration targets 61 and the liquid 60 that are stored in the reservoir part 30 are collected by using the collection tool 70. This provides ease of collecting the filtration targets 61. Cells that are to be taken out as filtration targets are collected together with the liquid and are thus protected from exposure to the atmosphere. This eliminates or reduces the possibility that the cells will become less active while being collected.

The filtration device 1A capable of collecting the filtration targets 61 together with the liquid 60 makes the process of collecting the filtration targets 61 less burdensome than if the filtration targets 61 exposed to the atmosphere are collected. Subsequent to filtration, continued exposure to the atmosphere can cause the filtration targets 61 to become deposited on the filtration device 1A, and as a result, the process of collecting the filtration targets 61 can become complicated. As a workaround, the filtration targets 61 having undergone filtration are immersed in the liquid 60. This may minimize the depositing of the filtration targets 61 on the filtration device 1A and provides ease of collecting the filtration targets 61.

Furthermore, minimization of the aggregation of an aggregable substance may be achieved through the use of the filtration device 1A. Centrifuges have conventionally used to collect cells from a cell suspension. During centrifugation, such a centrifuge exerts force (centrifugal force) in one direction in a manner so as to shorten the distance between particles of an aggregable substance, which are in turn more likely to come into contact with each other. Such a centrifugal process can cause the aggregation of the aggregable substance. For this reason, the centrifugation needs to be followed by a process of breaking up a mass of the aggregable substance. In the filtration device 1A, meanwhile, the filtration targets 61 having undergone filtration are immersed in the liquid 60. This minimizes aggregation of filtration targets and provides ease of collecting the filtration targets 61.

Another feature of the filtration device 1A is that the capacity in the space 51 of the reservoir part 30 is equal to the desired volume of liquid to be collected. Owing to this feature, any desired volume of the liquid 60 containing the filtration targets 61 may be collected. This eliminates the need for a process of weighing collected liquid.

The filtration device 1A has a simple configuration for cross-flow filtration. When getting deposited on the filtration part 20 during the filtration work, the filtration targets 61 may be washed downward by the liquid 60 introduced through the opening 13. The depositing of the filtration targets 61 on the filtration part 20 and clogging of the filtration part 20 may be minimized accordingly.

Adjusting the size of the through-holes 21 imparts selectivity to the filtration part 20; that is, living cells may be caught in the filtration part 20, and dead cells and/or dirt may pass through the filtration part 20. In this way, living cells are separated from dead cells and/or dirt. This gives an increase in the proportion of living cells in a cell suspension after a filtration session. Consequently, living cells may remain active over a prolonged period.

The filtration device 1A with minimized clogging of the filtration part 20 enables multiple filtration sessions. Specifically, Steps ST12 to ST15 may be repeated multiple times. The maximum treatable volume of the filtration targets 61 per filtration session is typically determined by the area of the filtration part 20. Nevertheless, an increase in the volume of the filtration targets 61 to be treated will not raise the possibility of clogging of the filtration device 1A, where the filtration targets 61 do not remain in the filtration part 20 and are retained in the reservoir part 30. This enables the filtration part 20 to remain serviceable after Step ST15, and the filtration device 1A can return to Step ST12 for another filtration session. Thus, the filtration device 1A alone can treat a large volume of the filtration targets 61.

The fluidity of liquid aids cells in moving from the reservoir part 30 to, for example, another receptacle. The physical strain on cells may be lighter than would be the case of transferring cells exposed to the atmosphere.

When cross sections of the reservoir part 30 are taken in directions (i.e., the X and Y directions) orthogonal to the direction of connection between the first end and the second end of the cylindrical body 10 (i.e., orthogonal to the Z direction), the opening cross-sectional area Sa2 of the (lower) portion of the reservoir part 30 close to the second end of the cylindrical body 10 is smaller than the opening cross-sectional area Sa1 of the (upper) portion of the reservoir part 30 close to the filtration part 20. This is conducive to drawing the filtration targets 61 and the liquid 60 into the lowermost end portion 32 of the reservoir part 30, from which the filtration targets 61 and the liquid 60 may be easily collected by using the collection tool 70. In particular, a channel with a small open tip, such as a tube, a needle, a pipette, or a syringe, may be selected as the collection tool 70. The filtration targets 61 and the liquid 60 in the reservoir part 30 may be mostly or entirely collected accordingly.

The inner wall 33 of the reservoir part 30 includes the inclined portion 35 inclined toward the second end of the cylindrical body 10. This is conducive to drawing the filtration targets 61 and the liquid 60 into the reservoir part 30, from which the filtration targets 61 and the liquid 60 may be easily collected.

The inclined portion 35 is inclined toward the center of the cylindrical body 10. This is more conducive to drawing the liquid 60 containing the filtration targets 61 into the lowermost end portion 32. The filtration targets 61 and the liquid 60 may thus be collected more easily by using the collection tool 70.

The outer wall 34 of the reservoir part 30 includes the protruding portion 36 protruding toward the second end of the cylindrical body 10. The liquid 62 drained out of the cylindrical body 10 from the filtration part 20 flows along the outer wall 34 of the reservoir part 30. This eliminates or reduces the possibility that the liquid 62 drained out of the cylindrical body 10 will splatter.

The side face of the protruding portion 36 is inclined toward the center of the cylindrical body 10. The liquid 62 drained from the filtration part 20 is thus led to the lowermost end portion 32. This eliminates or further reduces the possibility that the liquid 62 drained out of the cylindrical body 10 will splatter.

The filtration part 20 extends all around the circumferential portion 11 of the cylindrical body 10. That is, the filtration part 20 surrounds the circumferential portion 11 of the cylindrical body 10. This enables the filtration part 20 to improve drainage of the liquid 60 overflowing form the reservoir part 30 and is thus conducive to a short-time filtration.

The circumferential portion 11 of the cylindrical body 10 includes the frame members 14 that define the openings 15, each of which is an interface between the inside and the outside of the cylindrical body 10. The filtration part 20 is a cylindrical filter and is attached to the frame members 14. The filtration part 20 is easily provided on the circumferential portion 11 of the cylindrical body 10 accordingly. The cost of production may be lower than if the cylindrical body 10 and the filtration part 20 are formed as a single component.

The cylindrical body 10 is made of a resin through which the inside of the cylindrical body 10 is visible from outside the cylindrical body 10 (i.e., a transparent resin). This enables viewing of the filtration targets 61 and the liquid 60 that are stored in the reservoir part 30 and thus helps determine whether the reservoir part 30 is filled with the filtration targets 61 and the liquid 60.

The filtration part 20 is a filter made mainly of a metal and/or a metal oxide. This is conducive to a short-time filtration. Furthermore, the resulting ease of collecting the filtration targets 61 enables an increase in collection rate. Meanwhile, a resin filter has through-holes varying in size and arrangement, and as a result, filtration targets can get caught in the through-holes. A filter made mainly of a metal and/or a metal oxide is designed with through-holes that are more uniform in size and arrangement than through-holes of a resin filter. The filtration device 1A includes, as the filtration part 20, a filter made mainly of a metal and/or a metal oxide. Owing to this feature, the filtration targets 61 on the filtration part 20 can easily come off when being collected. Consequently, the collection rate may be higher than with the resin filter.

The filtration device 1A includes the liquid-retaining receptacle 40 close to the second end of the cylindrical body 10. The liquid 62, namely, the liquid drained out of the cylindrical body 10 from the filtration part 20 is retained in the liquid-retaining receptacle 40 accordingly.

As with the filtration device 1A, the filtration and collection method also produces the effects described above.

Embodiment 1 describes that the cylindrical body 10 has a cylindrical shape. In some embodiments, the cylindrical body 10 may, for example, be in the shape of a rectangular prism. Similarly, the shape of the filtration part 20 is not limited to a cylindrical shape. In some embodiments, the filtration part 20 may, for example, in the shape of a rectangular prism.

Embodiment 1 describes that the cylindrical body 10 is made of a resin through which the inside of the cylindrical body 10 is visible from outside the cylindrical body 10. In some embodiments, the cylindrical body 10 may be made of a resin through which the inside of the cylindrical body 10 is invisible from outside the cylindrical body 10.

Embodiment 1 describes that the cylindrical body 10 includes three frame members 14 and has three openings 15. It is only required that the cylindrical body 10 include at least one frame member 14 and have at least one opening 15. It is also described that the frame members 14 extend in the height direction of the cylindrical body 10. In some embodiments, however, the frame members 14 may extend in another direction; that is, the frame members 14 may be oblique.

Embodiment 1 describes that the filtration part 20 and the cylindrical body 10 are formed as discrete members. In some embodiments, the filtration part 20 may be integral with the cylindrical body 10. In this case, the frame members 14 of the cylindrical body 10 are optional.

Embodiment 1 describes that the inner wall 33 of the reservoir part 30 is dented so as to form a conical shape. In some embodiments, the inner wall 33 of the reservoir part 30 may be formed as a flat surface.

FIG. 11A schematically illustrates a filtration device 1AA according to a modification of Embodiment 1 of the present invention. As illustrated in FIG. 11A, the filtration device 1AA has a reservoir part 30aa with an inner wall 33aa, which is flat. In place of the recess at the center of the bottom portion, a recess is provided at an interface between a bottom face and a side face of the reservoir part 30aa. As in the embodiment above, liquid is drawn into the recess. This enables a reduction in the amount of residual liquid that remains after the collection of liquid. The reservoir part 30aa may have a tip shaped in conformance with the tip of a syringe needle that is to be used as the collection tool 70. This enables a further reduction in the amount of residual liquid.

FIG. 11B schematically illustrates a filtration device 1AB according to another modification of Embodiment 1 of the present invention. As illustrated in FIG. 11B, the filtration device 1AB includes a reservoir part 30ab and a valve 37, which is an open/close valve provided to a portion of the reservoir part 30ab. When the valve 37 is opened, a channel leads to the outside of the filtration device 1AB. Specifically, when the valve 37 is opened, the reservoir part 30ab communicates with the outside of the filtration device 1AB through a channel in a bottom portion of the reservoir part 30ab. With the turn of the valve, filtration targets and liquid that are in stored the reservoir part 30ab of the filtration device 1AB may be easily collected. Gravity helps reduce the amount of residual liquid that remains after the collection of liquid. The reason is as follows: the fluidity of liquid aids cells in moving out of the reservoir part 30ab, and the physical strain on cells may thus be lighter than would be the case of transferring cells exposed to the atmosphere.

With the valve 37 open, liquid that contains filtration targets may be introduced into the cylindrical body 10 of the filtration device 1AB from below. Subsequently, the valve 37 may be closed to store, in the reservoir part 30ab, the liquid that contains the filtration targets. The valve may be opened again to collect the filtration targets and the liquid. Consequently, the contents may be stirred in the filtration device 1AB.

Embodiment 1 describes that the outer wall 34 of the reservoir part 30 protrudes so as to form a conical shape. In some embodiments, the outer wall 34 of the reservoir part 30 may be formed as a flat surface.

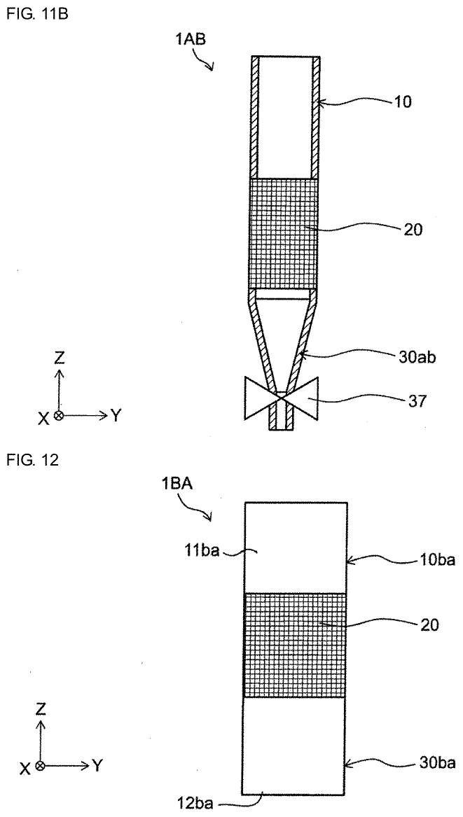

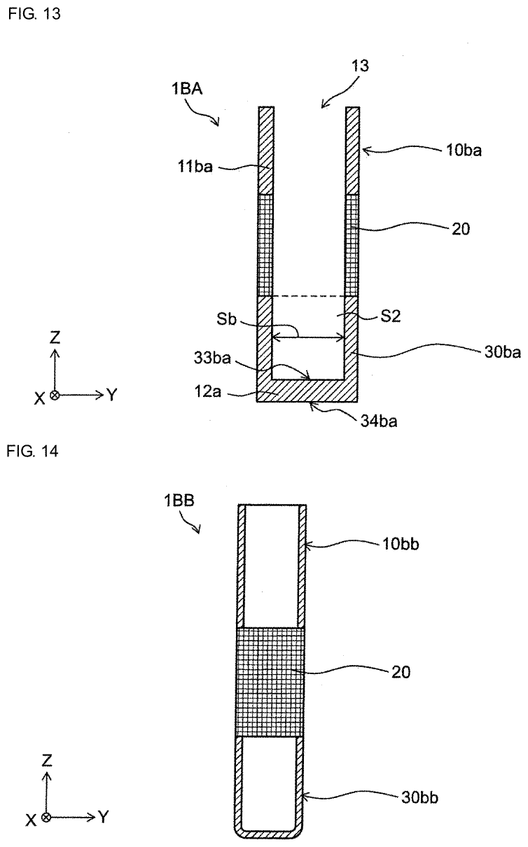

FIG. 12 schematically illustrates the configuration of a filtration device 1BA according to still another modification of Embodiment 1 of the present invention. FIG. 13 is a schematic sectional view of the filtration device 1BA according to the modification of Embodiment 1 of the present invention. As illustrated in FIGS. 12 and 13, the filtration device 1BA includes a cylindrical body 10ba, which has a bottom. The filtration part 20 is provided on a circumferential portion 11ba of the cylindrical body 10ba having a bottom. The filtration targets 61 and the liquid 60 may be stored in a reservoir part 30ba, which is located below the filtration part 20.

The reservoir part 30ba of the filtration device 1BA is defined by part of the circumferential portion 11ba of the cylindrical body 10ba and by an end wall 12ba. Specifically, the reservoir part 30ba is defined by part of the circumferential portion 11ba and the end wall 12ba, that is, by portions of the cylindrical body 10ba that are located below the filtration part 20.

The reservoir part 30ba has, on a bottom face thereof, an inner wall 33ba, which is formed as a flat surface extending in directions (i.e., the X and Y directions) orthogonal to the direction in which the circumferential portion 11ba extends (i.e., orthogonal to the Z direction). The reservoir part 30ba also has, on the bottom face thereof, an outer wall 34ba, which is formed as a flat surface extending in the directions (i.e., the X and Y directions) orthogonal to the direction in which the circumferential portion 11ba extends (i.e., orthogonal to the Z direction).

When cross sections of the reservoir part 30ba are taken in the directions (i.e., the X and Y directions) orthogonal to the height direction of the cylindrical body 10ba (i.e., orthogonal to the Z direction), an opening cross-sectional area Sb of the reservoir part 30ba is constant between an lower end of the filtration part 20 and the inner wall 33ba on the bottom face of the reservoir part 30ba.

A space S2 in the reservoir part 30ba of the filtration device 1BA may be larger than the space 51 of the reservoir part 30 of the filtration device 1A. The filtration device 1A and the filtration device 1BA are of the same height. Owing to the flatness of the inner wall 33ba on the bottom face of the reservoir part 30ba, the reservoir part 30ba achieves an increase in capacity in the space S2, and the volume of liquid that may be collected may be larger than would be possible with the filtration device 1A.

Owing to the flatness of the outer wall 34ba of the reservoir part 30ba, that is, owing to the flatness of the outer wall on the bottom face of the cylindrical body 10ba, the filtration device 1BA achieves the stability of the cylindrical body 10ba disposed in the liquid-retaining receptacle 40.

As described above, the inner wall 33ba and the outer wall 34ba of the reservoir part 30ba of the filtration device 1BA are each formed as a flat surface. In some embodiments, the inner wall 33ba of the reservoir part 30ba of the filtration device 1BA may be dented so as to form a conical shape, and the outer wall 34ba of the reservoir part 30ba may be formed as a flat surface. Alternatively, the inner wall 33ba of the reservoir part 30ba of the filtration device 1BA may be formed as a flat surface, and the outer wall 34ba of the reservoir part 30ba may protrude so as to form a conical shape.

FIG. 14 schematically illustrates a filtration device 1BB according to still another modification of Embodiment 1 of the present invention. As illustrated in FIG. 14, the filtration device 1BB includes a reservoir part 30bb, which has a flat inner wall with a curvature at an interface between a bottom face and a side face. This enables a reduction in the amount of residual liquid that remains in the reservoir part 30bb after the collection of liquid. Specifically, the curvature at the interface enables a reduction in the amount of residual liquid on the interface and also enable a reduction in the surface tension of the liquid on the interface.

Embodiment 1 describes that the filtration part 20 is a filter made of metal. In some embodiments, the filtration part 20 may be a membrane filter made of resin or any other filter through which the filtration targets 61 contained in the liquid 60 are taken out by filtration.

Embodiment 1 describes that the filtration part 20 extends all around the circumferential portion 11 of the cylindrical body 10; that is, the filtration part 20 surrounds the circumferential portion 11 of the cylindrical body 10. In some embodiments, the filtration part 20 may extend partially around the circumferential portion 11 of the cylindrical body 10. For example, the filtration part 20 may extend halfway or less around the circumferential portion 11 of the cylindrical body 10.

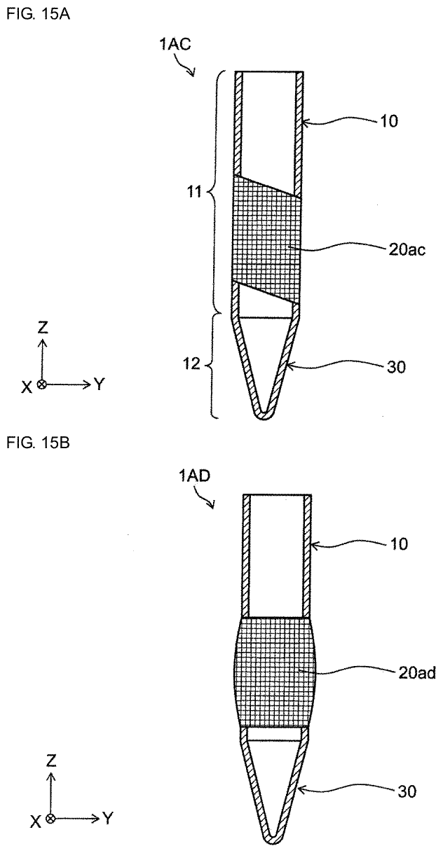

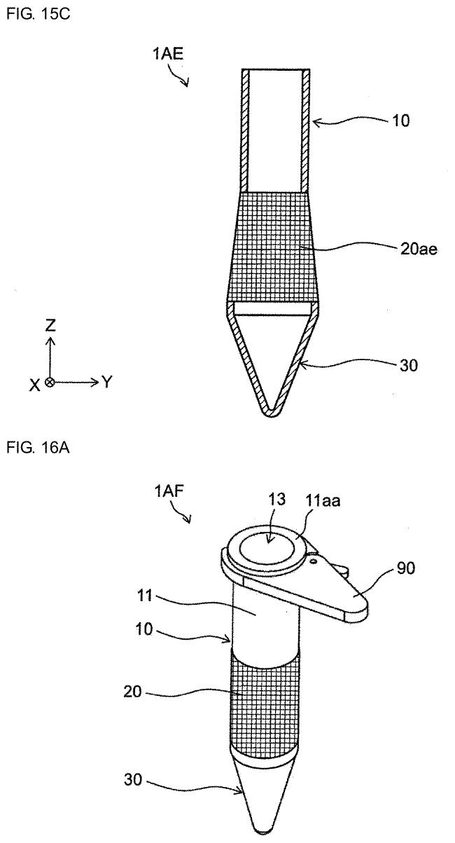

FIG. 15A schematically illustrates a filtration device 1AC according to still another modification of Embodiment 1 of the present invention. As illustrated in FIG. 15A, the filtration device 1AC includes a filtration part 20ac, part of which extends all around the circumferential portion 11 of the cylindrical body 10, with the rest extending only partially around the circumferential portion 11. For example, the filtration part 20ac of the filtration device 1AC is provided on the circumferential portion of the cylindrical body 10 and is oblique to the direction in which the cylindrical body 10 extends (i.e., oblique to the Z direction). Effects equivalent to the effects produced by the filtration device 1A may be attained accordingly.

FIG. 15B schematically illustrates a filtration device 1AD according to still another modification of Embodiment 1 of the present invention. As illustrated in FIG. 15B, the filtration device 1AD includes a filtration part 20ad, which is partially curved outward from the cylindrical body 10. When liquid is drained from the filtration part 20ad, a turbulent flow is likely to be produced on and around the outwardly curved portion. For this reason, this modification is advantageous in that the filtration targets are less likely to get deposited on the filtration part.

FIG. 15C schematically illustrates a filtration device 1AE according to still another modification of Embodiment 1 of the present invention. As illustrated in FIG. 15C, the filtration device 1AE includes a filtration part 20ae, the diameter of which increases in a direction toward the reservoir part 30. That is, the diameter of a lower portion of the filtration part 20ae is larger than the diameter of an upper portion of the filtration part 20ae. As with the aforementioned modification, this modification is advantageous in that filtration targets caught in the filtration part 20ae can readily settle by gravitation into the reservoir part 30 for storage. When liquid is drained from the filtration part 20ae, a turbulent flow is likely to be produced, as in the modification above. For this reason, this modification is advantageous in that the filtration targets are less likely to get deposited on the filtration part 20ae.

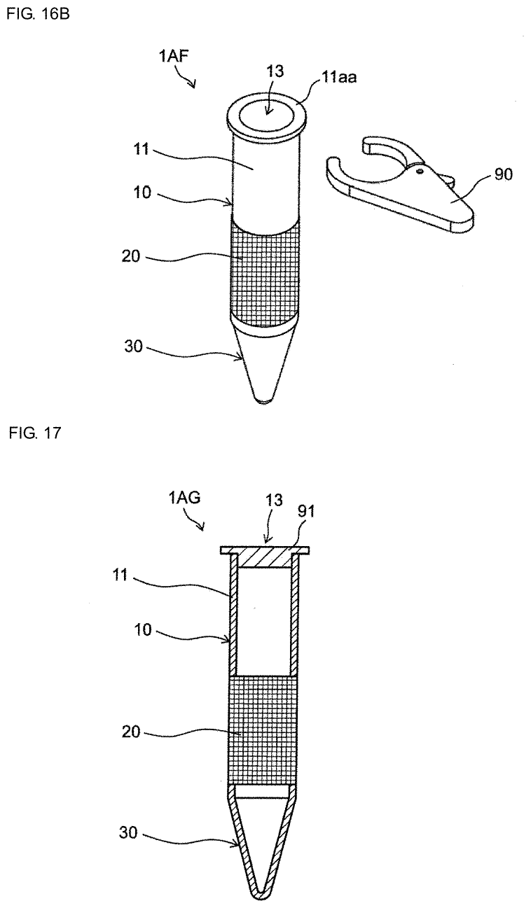

FIG. 16A schematically illustrates a filtration device 1AF according to still another modification of Embodiment 1 of the present invention. FIG. 16B is a schematic exploded view of the filtration device 1AF according to the modification of Embodiment 1 of the present invention. FIG. 16B illustrates the state in which a tab part 90 is removed from the filtration device 1AF. As illustrated in FIGS. 16A and 16B, the tab part 90 with which the filtration device 1AF is held is provided on the circumference of the cylindrical body 10 adjacent to the opening 13. The tab part 90 held by the user may keep the user's hand from direct contact with portions exposed to filtration targets. Contamination of the filtration targets may be minimized accordingly. The tab part 90 may be used to keep the device stationary in the liquid-retaining receptacle. This provides ease of handling. The tab part 90 may be integral with the cylindrical body 10 or may be detachable from the cylindrical body 10. Detaching the tab part 90 before putting the filtration device 1AF into storage results in space saving.

Referring to FIGS. 16A and 16B, the cylindrical body 10 is provided with a flange part 11aa, which extends along the circumference of the cylindrical body 10 adjacent to the opening 13. The flange part 11aa protrudes outward in the radial direction of the cylindrical body 10. The flange part 11aa is an auxiliary to the tab part 90. The flange part 11aa is in contact with the tab part 90 attached to the circumference of the cylindrical body 10. This eliminates or reduces the possibility that the filtration device 1AF will slip through the tab part 90.

FIG. 17 schematically illustrates a filtration device 1AG according to still another modification of Embodiment 1 of the present invention. As illustrated in FIG. 17, the filtration device 1AG is fitted with a lid 91, with which the opening 13 at the upper portion of the cylindrical body 10 is closed. The lid 91 reduces the possibility that filtration targets will become dry or become contaminated by, for example, the atmosphere. Subsequent to filtration, the lid 91 provides enhanced portability. The lid 91 may be detachable from the cylindrical body 10. Alternatively, the lid 91 may be a hinged lid that is partially fastened to the cylindrical body 10.

Embodiment 1 describes that the filtration device 1A is disposed in the liquid-retaining receptacle 40 before starting filtration. In some embodiments, the liquid-retaining receptacle 40 is optional. Instead of being disposed in the liquid-retaining receptacle 40, the filtration device 1A may be attached to another device before starting filtration. Alternatively, filtration by the filtration device 1A may be carried out without using the liquid-retaining receptacle 40.

Embodiment 1 describes that the filtration targets are cells and the liquid is a cell suspension. In some embodiments, filtration targets other than cells and a liquid other than a cell suspension may be used.

The filtration device 1A and the filtration method have been described as Embodiment 1, which is not limited thereto. For example, the filtration device 1A may be incorporated in a kit for implementing the filtration method.

### Embodiment 2

The following describes a filtration device according to Embodiment 2 of the present invention with a focus on differences between Embodiment 1 and Embodiment 2. Each component described in Embodiment 1 and the corresponding (identical or similar) component that will be described in Embodiment 2 are denoted by the same reference sign. Description that holds true for both Embodiments 1 and 2 will be omitted.

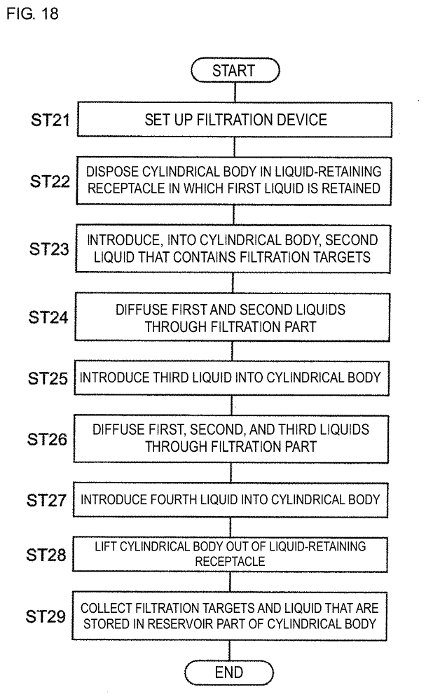

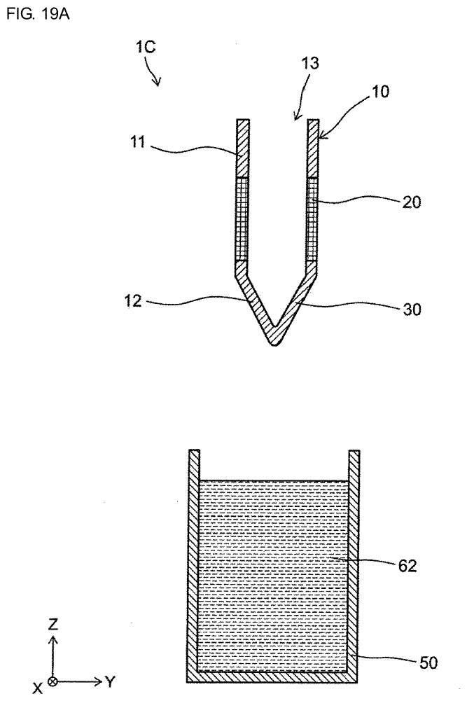

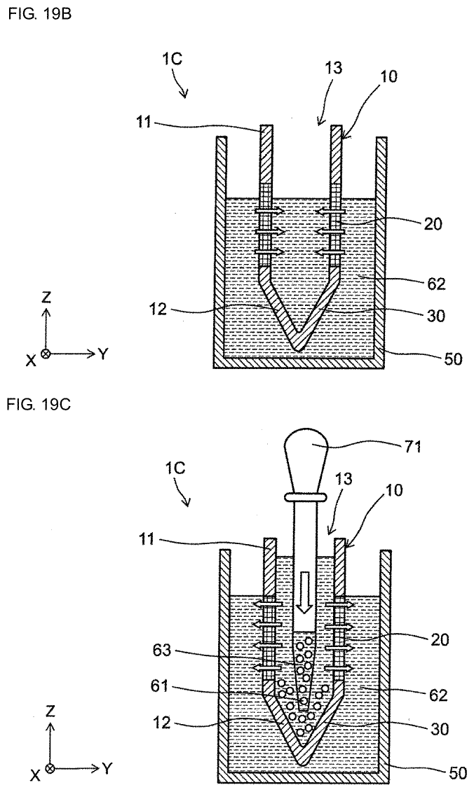

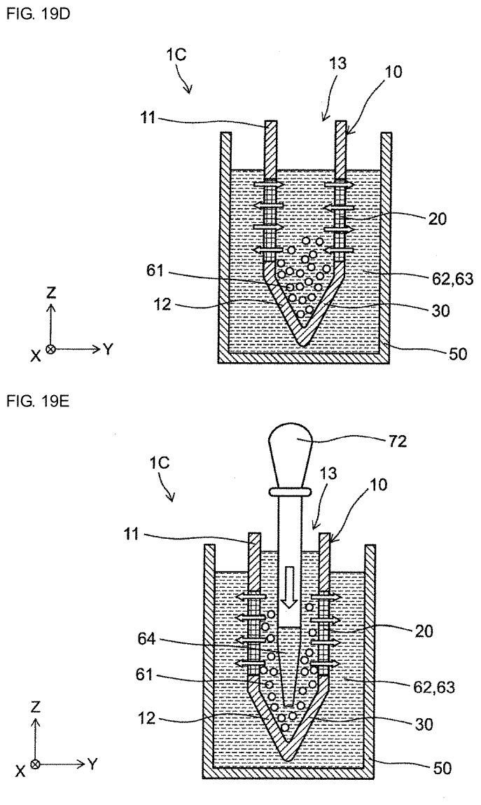

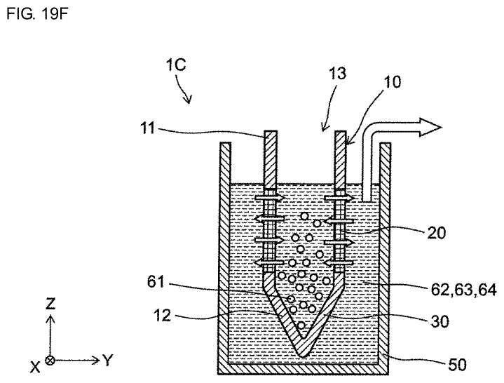

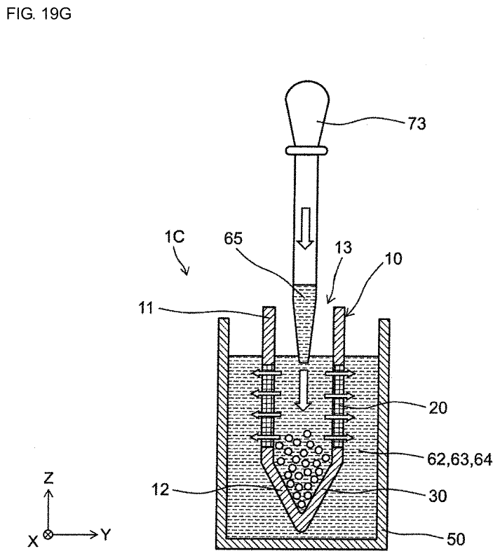

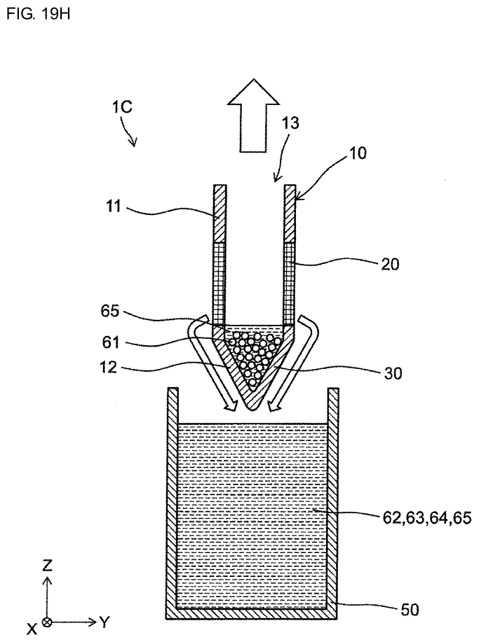

An example of a filtration method according to Embodiment 2 will be described below with reference to FIGS. 18 and 19A to 19I. FIG. 18 is a flowchart of an example of the filtration method according to Embodiment 2 of the present invention. FIGS. 19A to 19I illustrate steps that may be included in the filtration method according to Embodiment 2 of the present invention.

Embodiment 2 differs from Embodiment 1 in that filtration is carried out with the cylindrical body 10 immersed in the liquid 62.

Referring to FIG. 18 and FIG. 19A illustrating Step ST21, a filtration device 1C is set up. The filtration device 1C includes: the cylindrical body 10; the filtration part 20 on the circumferential portion 11 of the cylindrical body 10; the reservoir part 30 below the filtration part 20; and a liquid-retaining receptacle 50, in which a first liquid 62 is retained. The first liquid 62 in Embodiment 2 is phosphate buffered saline (PBS). The cylindrical body 10 is fixed to the liquid-retaining receptacle 50.

Referring to FIG. 18 and FIG. 19B illustrating Step ST22, the cylindrical body 10 is disposed in the liquid-retaining receptacle 50 in which the first liquid 62 is retained. In Step ST22, the cylindrical body 10 is immersed in the first liquid 62, which in turn passes through the filtration part 20 to flow into the cylindrical body 10. The liquid permeability of the through-holes 21 of the filtration part 20 is increased accordingly.

Referring to FIG. 18 and FIG. 19C illustrating Step ST23, a second liquid 63, together with the filtration targets 61 contained therein, is introduced into the cylindrical body 10. Specifically, a pipette 71 is inserted through the opening 13 of the cylindrical body 10 to introduce the second liquid 63 containing the filtration targets 61 into the cylindrical body 10. In Embodiment 2, the second liquid 63 is a cell suspension, and the filtration targets 61 are cells.

The second liquid 63 containing the filtration targets 61 is initially retained in the pipette 71. The pipette 71 is placed in such a manner that a tip thereof is close to the end wall 12 provided as a lower portion of the cylindrical body 10. In other words, the tip of the pipette 71 is placed within the reservoir part 30 in the lower portion of the cylindrical body 10. The second liquid 63 containing the filtration targets 61 is ejected from the tip of the pipette 71 and is introduced into the reservoir part 30 of the cylindrical body 10. Damage to the filtration targets 61 that is caused by the introduction of the second liquid 63 may be milder than if the second liquid 63 containing the filtration targets 61 is introduced in a manner so as to fall from the upper portion into the lower portion of the cylindrical body 10.

The second liquid 63 introduced into the cylindrical body 10 passes through the filtration part 20 to flow out of the cylindrical body 10.

Referring to FIG. 18 and FIG. 19D illustrating Step ST24, the first liquid 62 and the second liquid 63 diffuse through the filtration part 20. The first liquid 62 and the second liquid 63 are placed into suspension form as they pass through the filtration part 20 such that the surface of first liquid 62 becomes equal in level to the surface of the second liquid 63. Specifically, once the second liquid 63 containing the filtration targets 61 is ejected from the pipette 71 and is introduced into the cylindrical body 10, the filtration targets 61 are caught in the filtration part 20, and the second liquid 63 passes through the filtration part 20 to flow out of the cylindrical body 10. Consequently, the first liquid 62 retained in the liquid-retaining receptacle 50 is mixed with the second liquid 63 on the outside of the cylindrical body 10.

The first liquid 62 retained in the liquid-retaining receptacle 50 passes through the filtration part 20 to flow into the cylindrical body 10. Consequently, the first liquid 62 and the second liquid 63 are also mixed together in the cylindrical body 10.

In Step ST24, the first liquid 62 and the second liquid 63 diffuse as they pass through the filtration part 20. The depositing of the filtration targets 61 on the filtration part 20 may be minimized accordingly.

The introduction of the second liquid 63 containing the filtration targets 61 into the cylindrical body 10 in Step ST23 can possibly create a situation in which the surface of the liquid retained in the cylindrical body 10 is located at a level above the surface of the liquid retained in the liquid-retaining receptacle 50. In this case, the diffusion of the first liquid 62 and the second liquid 63 may be enabled by letting them stand until the surface of the liquid in the cylindrical body 10 becomes substantially equal in level to the surface of the liquid in the liquid-retaining receptacle 50.

Referring to FIG. 18 and FIG. 19E illustrating Step ST25, a third liquid 64 is introduced into the cylindrical body 10. In Step ST25, the third liquid 64 is introduced into the cylindrical body 10 to wash the filtration targets 61.

Specifically, a pipette 72 is inserted through the opening 13 of the cylindrical body 10 to introduce the third liquid 64 into the cylindrical body 10. The third liquid 64 in Embodiment 2 is a washing solution and may be PBS.

The third liquid 64 is initially retained in the pipette 72. The pipette 72 is placed in such a manner that a tip thereof is close to the end wall 12 provided as the lower portion of the cylindrical body 10. In other words, the tip of the pipette 72 is placed within the reservoir part 30 in the lower portion of the cylindrical body 10. The third liquid 64 is ejected from the tip of the pipette 72 and is introduced into the reservoir part 30 of the cylindrical body 10. Consequently, the filtration targets 61 are stirred in the cylindrical body 10, and the effect of washing may be enhanced accordingly.

Referring to FIG. 18 and FIG. 19F illustrating Step ST26, the first liquid 62, the second liquid 63, and the third liquid 64 diffuse through the filtration part 20. Specifically, the first liquid 62, the second liquid 63, and the third liquid 64 pass through the filtration part 20 to flow into and out of the cylindrical body 10. Consequently, the first liquid 62, the second liquid 63, and the third liquid 64 are mixed together.

In Embodiment 2, the liquids 62, 63, and 64 retained in the liquid-retaining receptacle 50 are partially recovered when the levels of the liquids 62, 63, and 64 rise and the surfaces of these liquids get close to the opening of the liquid-retaining receptacle 50. This will prevent the liquids 62, 63, and 64 from overflowing from the liquid-retaining receptacle 50.

Referring to FIG. 18 and FIG. 19G illustrating Step ST27, a fourth liquid 65 is introduced into the cylindrical body 10. Specifically, the fourth liquid 65 is ejected from a pipette 73 and is introduced into the cylindrical body 10. This causes the filtration targets 61 caught in the filtration part 20 to move into the reservoir part 30. The fourth liquid 65 is a recovery solution and may be PBS.

The tip of the pipette 73 is placed in the cylindrical body 10 and above the filtration part 20. The fourth liquid 65 is introduced into the side wall on the inside of the cylindrical body 10. The fourth liquid 65 causes the filtration targets 61 to come off from the filtration part 20 and to move into the reservoir part 30. An increase in the collection rate of the filtration targets 61 may be achieved accordingly.

Referring to FIG. 18 and FIG. 19H illustrating Step ST28, the cylindrical body 10 is lifted out of the liquid-retaining receptacle 50. Consequently, the fourth liquid 65 in the cylindrical body 10 passes through the filtration part 20 to flow out of the cylindrical body 10 and then moves downward. The filtration targets 61 and the remainder of the fourth liquid 65 are stored in the reservoir part 30.

Embodiment 2 involves shaking the cylindrical body 10 in side-to-side directions when lifting it out of the liquid-retaining receptacle 50. As a result, the filtration targets 61 come off from the filtration part 20 and are then stored in the reservoir part 30. An increase in the collection rate of the filtration targets 61 may be achieved accordingly.

Referring to FIGS. 18 and 19I illustrating Step ST29, the filtration targets 61 and the fourth liquid 65 that are stored in the reservoir part 30 of the cylindrical body 10 are collected. Specifically, the filtration targets 61 and the fourth liquid 65 that are stored in the reservoir part 30 are collected by using the collection tool 70.

The filtration device 1C and the filtration method according to Embodiment 2 produce the following effects.

According to the filtration method implemented with the filtration device 1C, filtration is carried out while the cylindrical body 10 is in contact with the first liquid 62 retained in the liquid-retaining receptacle 50. This contributes to the enhanced filtration efficiency. Specifically, the depositing of the filtration targets 61 on the filtration part 20 may be minimized, and an increase in the collection rate of the filtration targets 61 may be achieved accordingly.

A liquid-stirring mechanism such as a stirrer, a rotary screw, or a vibration mechanism may be provided in the liquid-retaining receptacle 50 to help minimize the depositing of the filtration targets on the filtration part. Alternatively, the cylindrical body may be vibrated or rotated. A further increase in the collection rate of filtration targets may be achieved accordingly.

Cells that are to be taken out as the filtration targets 61 are protected from exposure to the atmosphere, and the cells may thus remain active.

Adjusting the size of the through-holes 21 imparts selectivity to the filtration part 20; that is, living cells may be caught in the filtration part 20, and dead cells and/or dirt may pass through the filtration part 20. In this way, living cells are separated from dead cells and/or dirt.

The filtration method involves immersing the cylindrical body 10 in the first liquid 62. The liquid permeability of the through-holes 21 of the filtration part 20 may be increased accordingly.

The filtration method also involves placing the pipette 73 in the reservoir part 30 to introduce the second liquid 63 containing the filtration targets 61 into the cylindrical body 10. Damage to the filtration targets 61 may be milder than if the second liquid 63 is introduced from the upper portion of the cylindrical body 10.

The filtration method also involves placing the pipette 73 in the reservoir part 30 to introduce the third liquid 64, namely, a washing solution into the cylindrical body 10. Consequently, a buildup of the filtration targets 61 in the reservoir part 30 is stirred, and the effect of washing may be enhanced accordingly.

The filtration method also involves introducing, into the cylindrical body 10 immersed in the liquid, the fourth liquid 65, namely, a recovery solution before collecting the filtration targets 61. The filtration targets 61 caught in the filtration part 20 are prompted to move to the lower portion of the cylindrical body 10, and consequently, the filtration targets 61 are stored in the reservoir part 30. An increase in the collection rate of the filtration targets 61 may be achieved accordingly.

Embodiment 2 describes that the second liquid 63, the third liquid 64, and the fourth liquid 65 are introduced into the cylindrical body 10 by using the pipettes 71, 72, and 73, respectively. However, the tools that may be used to introduce the second liquid 63, the third liquid 64, and the fourth liquid 65 are not limited to the pipettes 71, 72, and 73. In some embodiments, the second liquid 63, the third liquid 64, and the fourth liquid 65 may be introduced by using syringes or tubes.

Embodiment 2 describes that the tips of the pipettes 72 and 73 are placed in the reservoir part 30 to introduce the second liquid 63 and the third liquid 64, respectively. In some embodiments, the tips of the pipettes 72 and 73 may be placed above the reservoir part 30.

Embodiment 2 describes that Step ST21 is followed by Step ST22. In some embodiments, the cylindrical body 10 may be disposed in the liquid-retaining receptacle 50 before introducing the first liquid 62 into the liquid-retaining receptacle 50 and the cylindrical body 10.

Embodiment 2 describes that the liquids retained in the liquid-retaining receptacle 50 are partially recovered in Step ST26. In some embodiments, the partial recovery of the liquids retained in the liquid-retaining receptacle 50 may be performed in another step. The partial recovery of the liquid is optional.

Embodiment 2 describes that the filtration method includes Step ST27 in which the fourth liquid 65, namely, a recovery solution is introduced into the cylindrical body 10. In some embodiments, Step ST27 of the filtration method may be skipped.

### Embodiment 3

The following describes a filtration system according to Embodiment 3 of the present invention with a focus on differences between Embodiment 1 and Embodiment 3. Each component described in Embodiment 1 and the corresponding (identical or similar) component that will be described in Embodiment 3 are denoted by the same reference sign. Description that holds true for both Embodiments 1 and 3 will be omitted.

The filtration device 1A according to Embodiment 1 is incorporated in an example of the filtration system according to Embodiment 3.

[Overall Configuration]

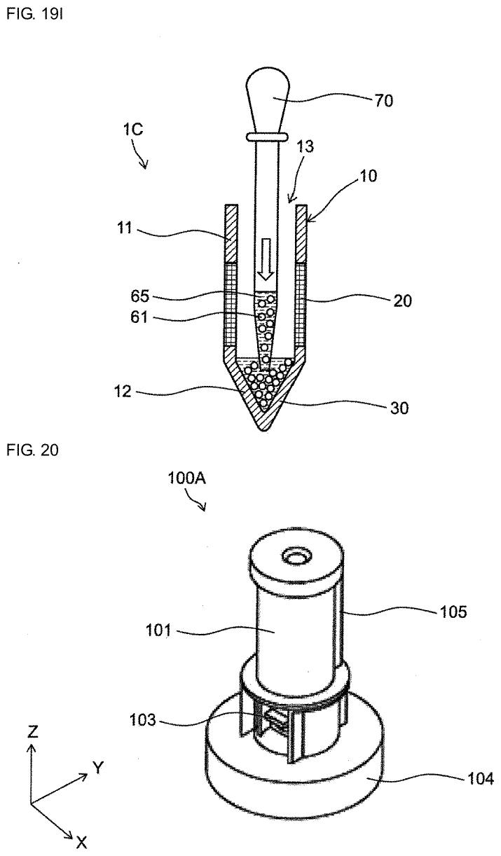

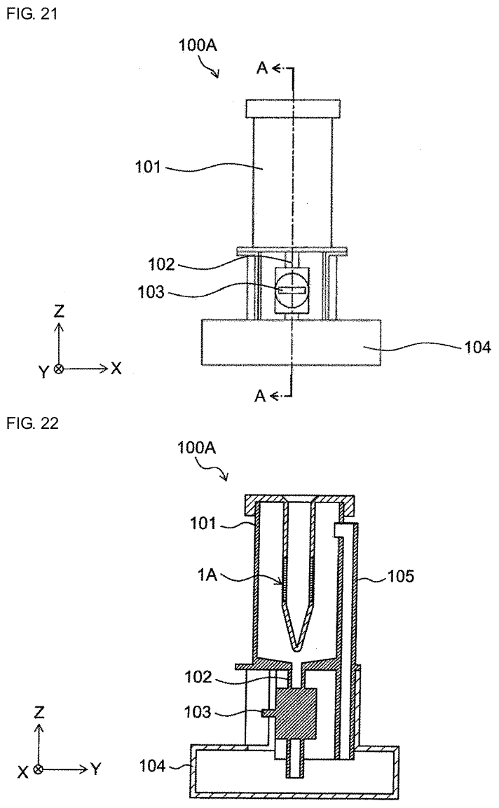

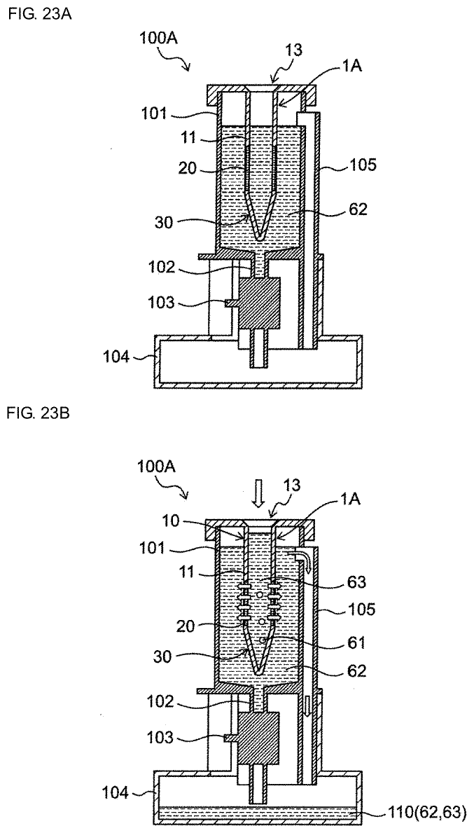

FIG. 20 is a schematic perspective view of an example of a filtration system 100A according to Embodiment 3 of the present invention. FIG. 21 is a schematic front view of the filtration system 100A according to Embodiment 3 of the present invention. FIG. 22 is a schematic sectional view of the filtration system 100A taken along line A-A in FIG. 21.

As illustrated in FIGS. 20 to 22, the filtration system 100A includes the filtration device 1A, a liquid-retaining receptacle 101, a channel 102, a valve 103, a waste liquid receptacle 104, and a waste liquid channel 105.

The filtration device 1A is disposed in the liquid-retaining receptacle 101. The filtration device 1A is as has already been described in Embodiment 1 and will not be further elaborated here.

The liquid-retaining receptacle 101 is a cylindrical receptacle having a bottom. The bottom portion of the liquid-retaining receptacle 101 has a vertically downward slope extending toward the center. The channel 102 is provided to the center of the bottom portion of the liquid-retaining receptacle 101 and extends toward the waste liquid receptacle 104. Liquid retained in the liquid-retaining receptacle 101 flows toward the channel 102 provided to the center of the bottom portion of the liquid-retaining receptacle 101.

The channel 102 is a path that connects the liquid-retaining receptacle 101 to the waste liquid receptacle 104. One end of the channel 102 is connected to the center of the bottom portion of the liquid-retaining receptacle 101. The other end of the channel 102 is located in the waste liquid receptacle 104. The channel 102 extends downward in the vertical direction from the center of the liquid-retaining receptacle 101 and is connected to the waste liquid receptacle 104. The liquid retained in the liquid-retaining receptacle 101 flows through the channel 102 into the waste liquid receptacle 104.

The valve 103 is provided on the channel 102. The flow of the liquid from the liquid-retaining receptacle 101 to the waste liquid receptacle 104 is controlled by opening or closing the valve 103. Specifically, opening the valve 103 allows the liquid to flow from the liquid-retaining receptacle 101 to the waste liquid receptacle 104. The flow of the liquid from the liquid-retaining receptacle 101 to the waste liquid receptacle 104 is dammed up by closing the valve 103.

The liquid in the liquid-retaining receptacle 101 flows through the channel 102 and is retained in the waste liquid receptacle 104. The waste liquid receptacle 104 is disposed below the liquid-retaining receptacle 101.

The waste liquid channel 105 is a path that connects the liquid-retaining receptacle 101 to the waste liquid receptacle 104. One end of the waste liquid channel 105 is located above the filtration part 20 of the filtration device 1A and is connected to a side wall of the liquid-retaining receptacle 101. The other end of the waste liquid channel 105 is located in the waste liquid receptacle 104. The liquid retained in the liquid-retaining receptacle 101 can flow through the waste liquid channel 105 into the waste liquid receptacle 104. This will prevent the liquid from overflowing from the liquid-retaining receptacle 101.

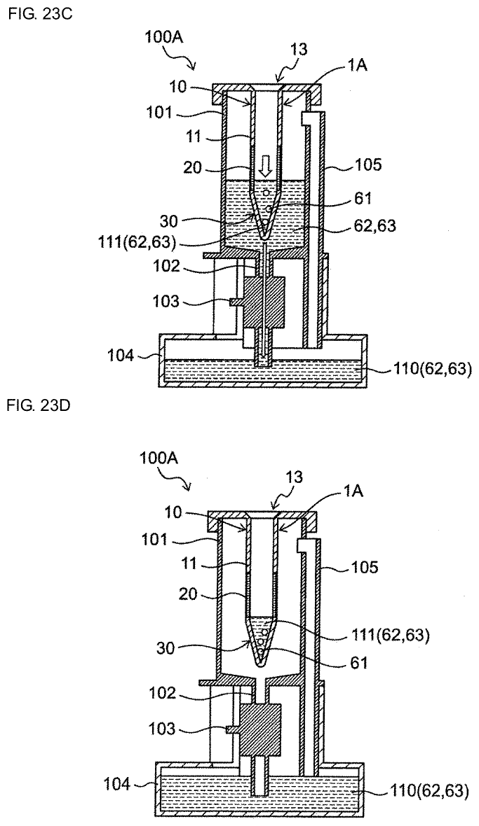

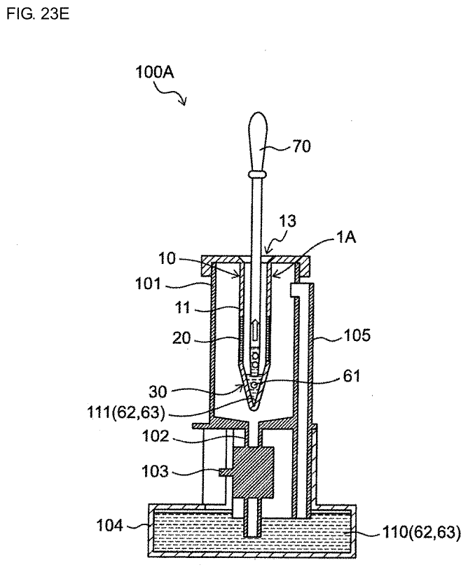

Actions that may be performed by the filtration system 100A will be described below with reference to FIGS. 23A to 23E. FIGS. 23A to 23E illustrates actions that may be performed by the filtration system 100A according to Embodiment 3 of the present invention.

Referring to FIG. 23A, the filtration system 100A is set up. Specifically, the filtration device 1A is disposed in the liquid-retaining receptacle 101 in which the first liquid 62 is retained.

Referring to FIG. 23B, the second liquid 63 containing the filtration targets 61 is introduced into the cylindrical body 10 through the opening 13 of the cylindrical body 10. The second liquid 63 introduced into the cylindrical body 10 passes through the filtration part 20 to flow out of the cylindrical body 10. Consequently, the first liquid 62 and the second liquid 63 are mixed together in the liquid-retaining receptacle 101.

As the second liquid 63 passes through the filtration part 20 to flow out of the cylindrical body 10, the amount of liquid in the liquid-retaining receptacle 101 increases, with an equivalent amount of liquid being discharged into the waste liquid receptacle 104 through the waste liquid channel 105. The liquids 62 and 63 flow out of the liquid-retaining receptacle 101 through the waste liquid channel 105 and are then stored as a waste liquid 110 in the waste liquid receptacle 104. This will prevent the liquids from overflowing from the liquid-retaining receptacle 101. Referring to FIG. 23B, the valve 103 on the channel 102 is closed.

Referring to FIG. 23C, the valve 103 is opened to allow the liquids 62 and 63 in the liquid-retaining receptacle 101 to flow through the channel 102 into the waste liquid receptacle 104. A liquid 111 in the cylindrical body 10 passes through the filtration part 20 to flow out of the cylindrical body 10. The liquid 111 remaining in the cylindrical body 10 contains the filtration targets 61 and increasingly becomes concentrated in the reservoir part 30.

Referring to FIG. 23D, the surface of the liquid 111 in the cylindrical body 10 comes down to the lower end of the filtration part 20, where the liquid 111 does not flow anymore out of the cylindrical body 10 through the filtration part 20. As a result, the concentrated liquid 111 is stored in the reservoir part 30.

Referring to FIG. 23E, the filtration targets 61 and the liquid 111 that are stored in the reservoir part 30 are collected. Specifically, the filtration targets 61 and the liquid 111 that are stored in the reservoir part 30 are collected by using the collection tool 70.

The filtration system 100A according to Embodiment 3 produces the following effects.

The filtration system 100A is distinctive in that the channel 102 connecting the bottom portion of the liquid-retaining receptacle 101 to the waste liquid receptacle 104 is provided with the valve 103. The flow of the liquid from the liquid-retaining receptacle 101 to the waste liquid receptacle 104 is controlled by opening or closing the valve 103. Opening and closing the valve 103 of the filtration system 100A enables the liquid in the cylindrical body 10 to become concentrated. This is easier than Step ST28 of the filtration method according to Embodiment 2, in which the cylindrical body 10 is lifted out of the liquid-retaining receptacle (see FIG. 19H).

The filtration system 100A performs standardized operations for causing filtration targets to come off, and the unevenness in collection rate may be reduced accordingly.

The cylindrical body 10 in Embodiment 3 is lidless at the opening 13. To allow for aseptic practices, the cylindrical body 10 may be fitted with a lid part provided to the opening 13 and having a closed channel, through which liquid can flow into and out of the cylindrical body 10. Similarly, the liquid-retaining receptacle 101 and the waste liquid receptacle 104 may have closed channels. Alternatively, an aseptic filter (e.g., a membrane filter with a pore size of 0.22 μm) may be provided to the opening 13 or part of the closed channel. The pressure in each receptacle may be controlled, or liquid may be caused to flow into and out of the receptacle accordingly. The term “closed channel” herein refers a channel having a side wall that keeps an inflow and an outflow of liquid from contact with outside air. The closed channel may be, for example, a tube.

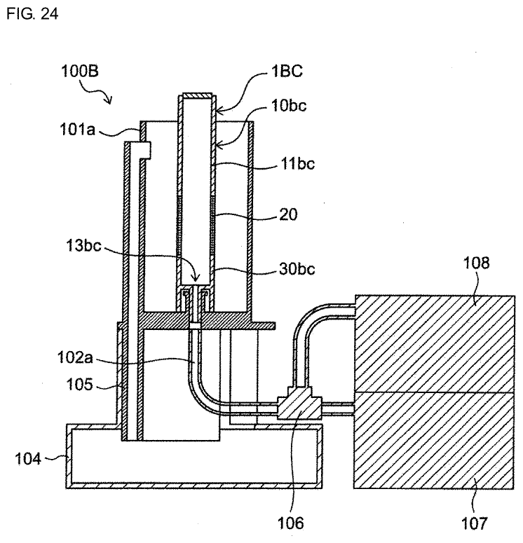

FIG. 24 schematically illustrates a filtration system 100B according to a modification of Embodiment 3 of the present invention. The filtration system 100B may include a liquid-feeding mechanism (e.g., a pump), which is omitted from FIG. 24. As illustrated in FIG. 24, the filtration system 100B includes a filtration device 1BC, a liquid-retaining receptacle 101a, a channel 102a, the waste liquid receptacle 104, the waste liquid channel 105, a switching valve 106, a sample receptacle 107, and a collection receptacle 108. Description that holds true for both the filtration system 100A according to Embodiment 3 and the filtration system 100B will be omitted.

The filtration device 1BC has a closed upper end and includes a cylindrical body 10bc, the filtration part 20, and a reservoir part 30bc. The cylindrical body 10bc has an opening 13bc, which is at a lower end of the cylindrical body 10bc. The filtration part 20 is provided on a circumferential portion 11bc of the cylindrical body 10bc. The reservoir part 30bc is located below the filtration part 20.

The reservoir part 30bc is defined by a lower portion of the cylindrical body 10bc, that is, by the portion located below the filtration part 20. Specifically, the reservoir part 30bc is defined by a side wall and a bottom portion of the cylindrical body 10bc that are located below the filtration part. The opening 13bc for inflow and outflow of liquid is provided at a bottom portion of the reservoir part 30bc. The channel 102a leads to the opening 13bc.

The filtration device 1BC is disposed in the liquid-retaining receptacle 101a.

The channel 102a is composed of a first channel and a second channel. The first channel connects the filtration device 1BC to the sample receptacle 107, and the second channel connects the filtration device 1BC to the collection receptacle 108. Switching between the first channel and the second channel is performed with the switching valve 106.

The sample receptacle 107 is a receptacle in which liquid containing filtration targets is retained. After undergoing the filtration by the filtration device 1BC, the filtration targets and the liquid are collected in the collection receptacle 108.

The filtration system 100B is configured in such a manner that the liquid containing the filtration targets and stored in the sample receptacle 107 is taken in from the bottom portion of the cylindrical body 10bc of the filtration device 1BC and is introduced into the cylindrical body 10bc. The switching valve 106 switches the channel 102a to the first channel, which connects the sample receptacle 107 to the filtration device 1BC.

A pump may be used to cause the liquid containing the filtration targets and stored in the sample receptacle 107 to flow through the channel 102a, or more specifically, through the first channel. The liquid flowing through the channel 102a is taken in from the bottom portion of the filtration device 1BC and is introduced into the cylindrical body 10bc. The liquid introduced into the cylindrical body 10bc passes through the filtration part 20 to flow out of the cylindrical body 10bc and is then stored in the liquid-retaining receptacle 101a. Filtration is carried out in this manner, and as a result, the liquid containing the filtration targets is condensed in the reservoir part 30bc.

This operation is herein referred to as a first operation α, which includes causing the liquid containing the filtration targets and stored in the sample receptacle 107 to flow through the first channel, taking in the liquid from the bottom portion of the cylindrical body 10bc of the filtration device 1BC, and introducing the liquid into the cylindrical body 10bc.

Subsequent to the filtration carried out by the filtration device 1BC, the switching valve 106 is turned so as to switch the channel 102a to the second channel, which connects the collection receptacle 108 to the filtration device 1BC.

A pump may be used to cause the filtration targets and the liquid that are stored in the reservoir part 30bc of the filtration device 1BC to flow into the collection receptacle 108 through the channel 102a, or more specifically, through the second channel. Consequently, the liquid condensed in the reservoir part 30bc of the filtration device 1BC is collected and stored in the collection receptacle 108.

This operation is herein referred to as a second operation (3, which includes collecting the filtration targets and the liquid that are stored in the reservoir part 30bc of the filtration device 1BC by causing them to flow through the second channel and storing them in the collection receptacle 108.

The filtration system 100B is capable of performing the first operation α and the second operation β alternately and consecutively. The area of the filtration part 20, the capacity of the reservoir part 30b, the capacity of the liquid-retaining receptacle 101a, the capacity of the waste liquid receptacle 104, properties (e.g., viscosity and aggregability) of filtration targets and liquid, and other factors place an upper limit to the throughput (i.e., the volume of liquid that contains filtration targets, the volume of filtration targets, or the concentration of filtration targets). Although the target throughput may not be reached in a single cycle of the first operation α and the second operation β, repeated cycles of the first operation α and the second operation β make the target throughput achievable in a closed environment. The filtration system 100B may include an additional valve on the channel 102a or an additional receptacle to perform complicated tasks by using various liquid.

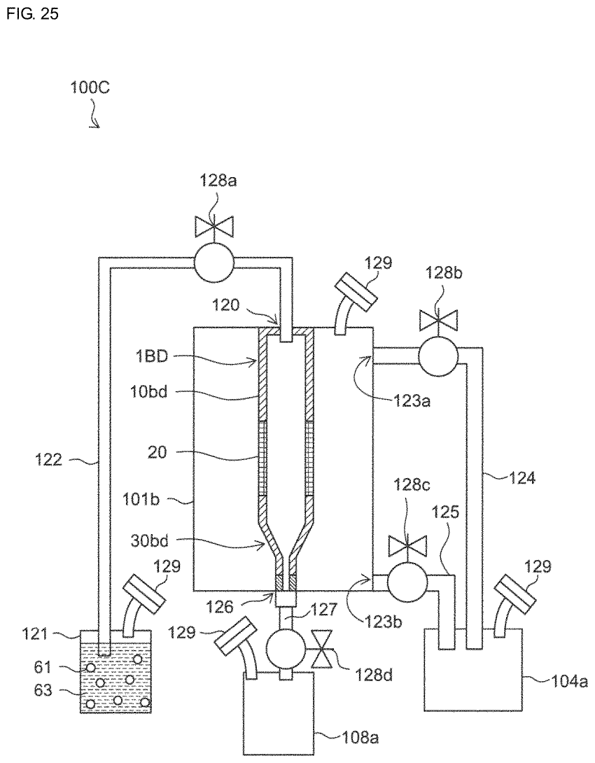

FIG. 25 schematically illustrates a filtration system 100C according to a modification of Embodiment 3 of the present invention. A filtration device 1BD in FIG. 25 is shown in cross section for easy-to-understand illustration. As illustrated in FIG. 25, the filtration system 100C includes the filtration device 1BD, a liquid-retaining receptacle 101b, a channel 122, a waste liquid receptacle 104a, a channel 124, a channel 125, a collection receptacle 108a, a channel 127, a valve 128a, a valve 128b, a valve 128c, and a valve 128d. The channel 122 connects a feed opening 120 of the filtration device 1BD to a receptacle 121, in which a cell suspension is stored. The channel 124 connects a first waste liquid outlet 123a of the liquid-retaining receptacle 101b to the waste liquid receptacle 104a. The channel 125 connects a second waste liquid outlet 123b of the liquid-retaining receptacle 101b to the waste liquid receptacle 104a. The channel 127 connects a collection port 126 of the filtration device 1BD to the collection receptacle 108a. The valves 128a, 128b, 128c, and 128d are provided on the channels 122, 124, 125, and 127, respectively. The cell suspension in the example illustrated in FIG. 25 is the liquid 63 in which cells are contained as the filtration targets 61.

The filtration system 100C is a closed system that is connected to outside air through only filters 129. The pressure in the closed system may be regulated through the filters 129. For example, the liquid-retaining receptacle 101b, the receptacle 121, the waste liquid receptacle 104a, and the collection receptacle 108a are connected with their respective filters 129.

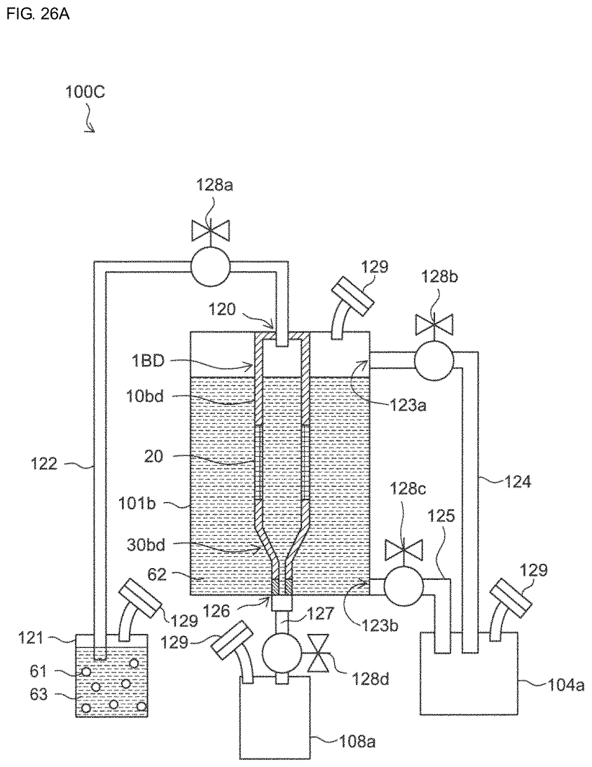

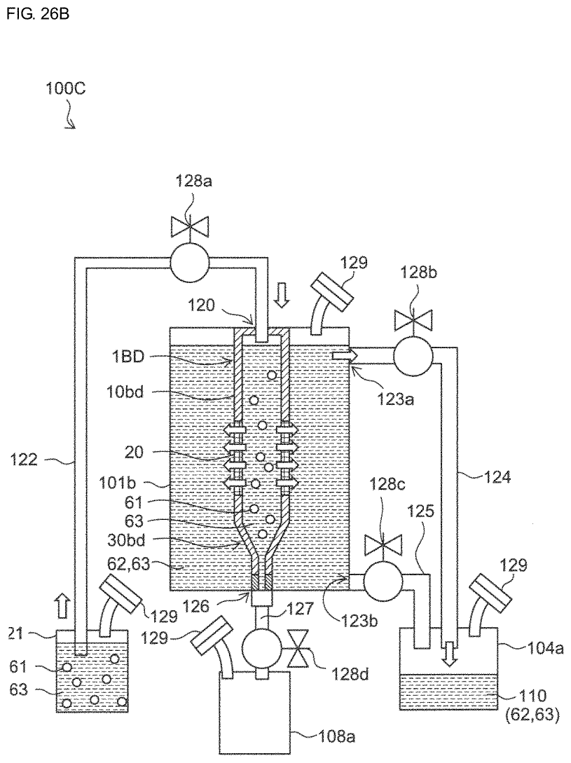

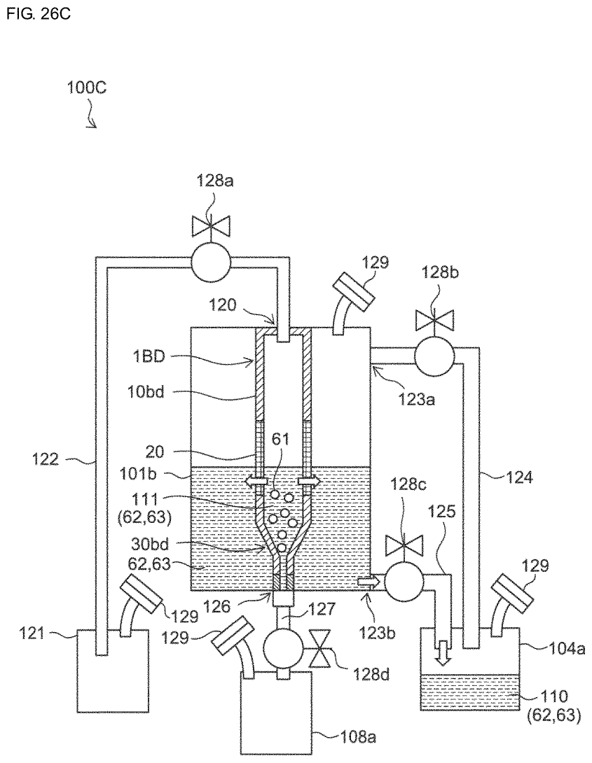

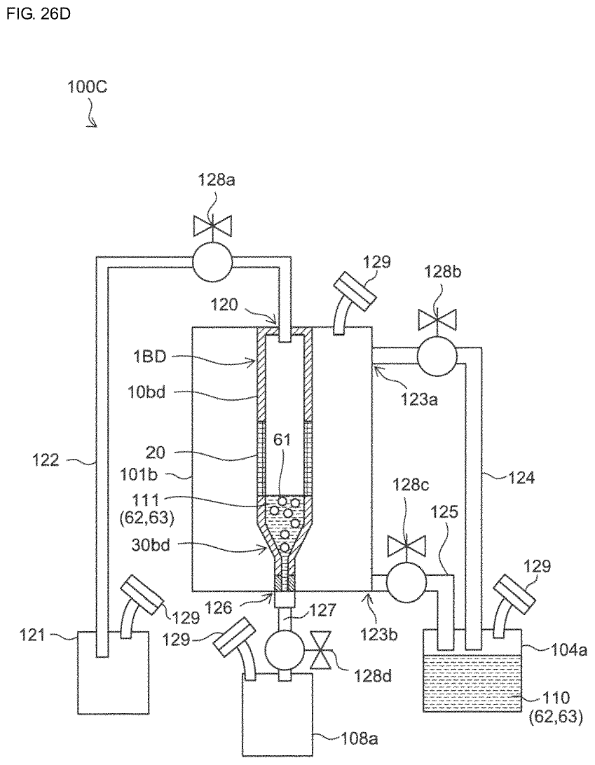

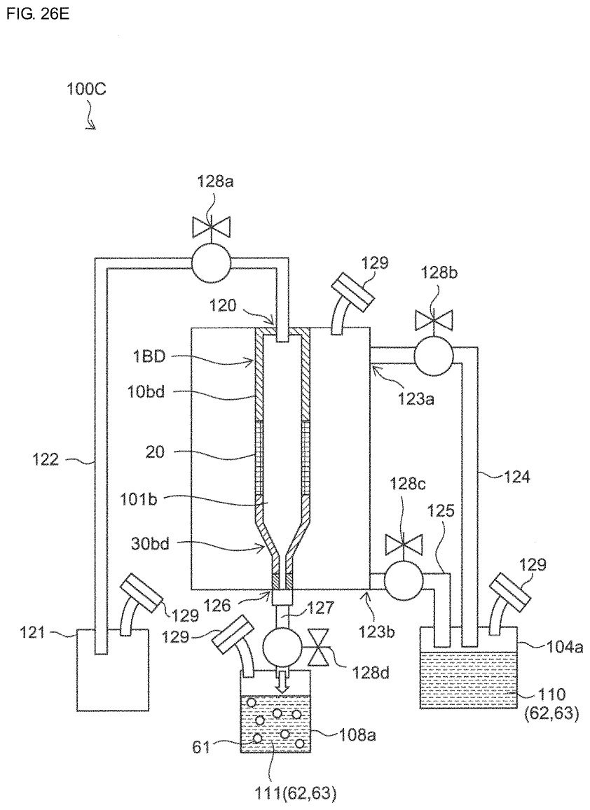

FIGS. 26A to 26E illustrate actions that may be performed by the filtration system 100C according to the modification of Embodiment 3 of the present invention. The filtration device 1BD in FIG. 26A to 26E is shown in cross section for easy-to-understand illustration. Referring to FIG. 26A, the first liquid 62 is retained in the liquid-retaining receptacle 101b as in the case with the filtration system 100B (see FIG. 23A). That is, the first liquid 62 is introduced into the liquid-retaining receptacle 101b before a cell suspension is filtered by the filtration system 100B.

Referring to FIG. 26B, the second liquid 63 containing the filtration targets 61 is introduced into the filtration device 1BD through the feed opening 120 of the filtration device 1BD in a state in which the feed valve 128a is open and the collection valve 128d is closed. The second liquid 63 containing the filtration targets 61 and stored in the receptacle 121 is fed into a cylindrical body 10bd through the feed opening 120 of the filtration device 1BD by using, for example, a pump. The second liquid 63 passes through the filtration part 20 to flow out of the cylindrical body 10bd. As the amount of the liquids 62 and 63 in the liquid-retaining receptacle 101b increases, an equivalent amount of liquid is drawn into the waste liquid receptacle 104a, with the first waste liquid valve 128b left open. The excess liquids 62 and 63 are stored as the waste liquid 110 in the waste liquid receptacle 104a.

Referring to FIG. 26C, the second waste liquid valve 128c is opened to allow the liquids 62 and 63 in the liquid-retaining receptacle 101b to flow into the waste liquid receptacle 104a. The liquid 111 in the cylindrical body 10bd passes through the filtration part 20 to flow out of the cylindrical body 10bd. The liquid 111 in the cylindrical body 10bd contains the filtration targets 61 and increasingly becomes concentrated in a reservoir part 30bd.

Referring to FIG. 26D, the surface of the liquid 111 in the cylindrical body 10bd comes down to the lower end of the filtration part 20, and the collection valve 128d is then opened. Referring to FIG. 26E, the filtration targets 61 and the liquid 111 flow out of the reservoir part 30bd into the collection receptacle 108a accordingly.

### Embodiment 4

The following describes a filtration device according to Embodiment 4 of the present invention with a focus on differences between Embodiment 1 and Embodiment 4. Each component described in Embodiment 1 and the corresponding (identical or similar) component that will be described in Embodiment 4 are denoted by the same reference sign. Description that holds true for both Embodiments 1 and 4 will be omitted.

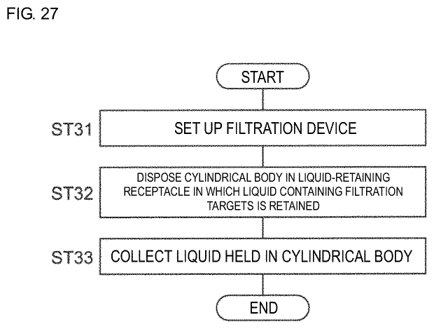

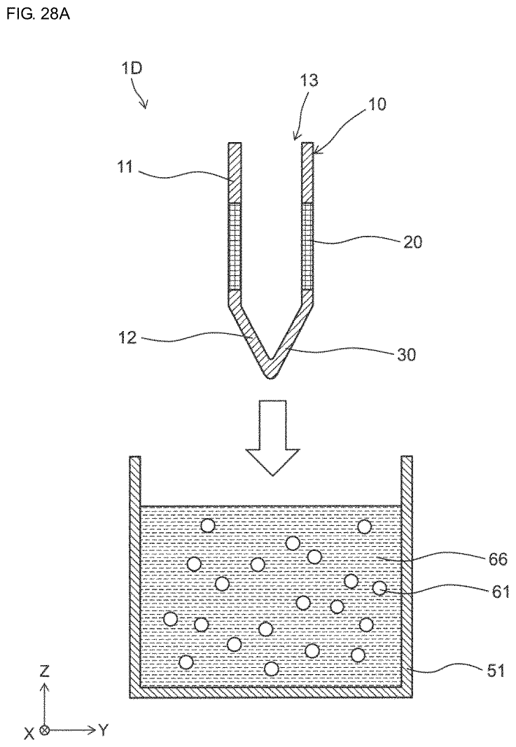

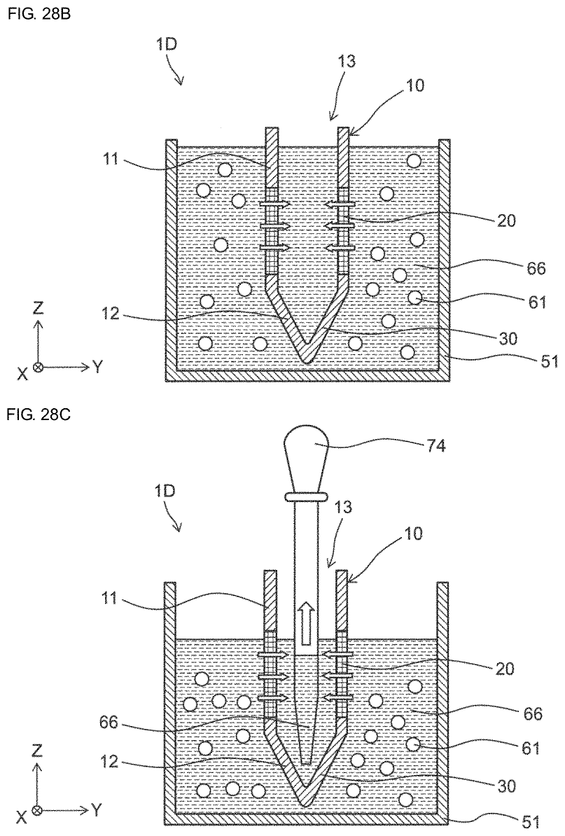

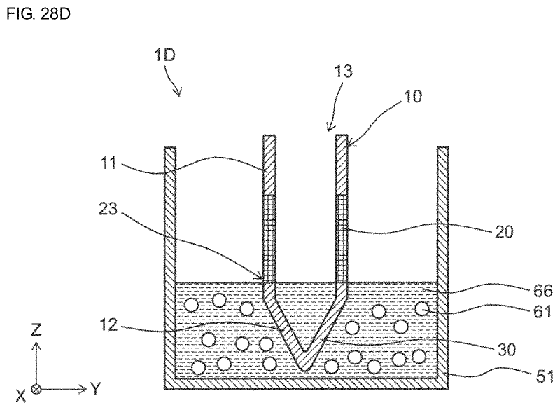

An example of a filtration method according to Embodiment 4 will be described below with reference to FIGS. 27 and 28A to 28D. FIG. 27 is a flowchart of an example of the filtration method according to Embodiment 4 of the present invention. FIGS. 28A to 28D illustrate steps that may be included in the filtration method according to Embodiment 4 of the present invention.

Embodiment 4 differs from Embodiment 1 in that filtration is carried out with the cylindrical body 10 immersed in a liquid 66, which contains the filtration targets 61. The term “filtration” herein also implies concentration. The term “concentration” as used hereinafter means that the proportion of the filtration targets 61 contained in the liquid 66 is increased. The filtration device and the filtration method according to Embodiment 4 may also be referred to as a concentration device and a concentration method, respectively.

Referring to FIG. 27 and FIG. 28A illustrating Step ST31, a filtration device 1D is set up. The filtration device 1D includes: the cylindrical body 10; the filtration part 20 on the circumferential portion 11 of the cylindrical body 10; the reservoir part 30 below the filtration part 20; and a liquid-retaining receptacle 51, in which the liquid 66 containing the filtration targets 61 is retained. In Embodiment 4, the liquid 66 is a cell suspension, and the filtration targets 61 are cells.

The cylindrical body 10 in Embodiment 4 is fixed to the liquid-retaining receptacle 51. The liquid-retaining receptacle 51 is a beaker, a test tube, a tank, or any other receptacle in which the liquid 66 may be retained.

Referring to FIG. 27 and FIG. 28B illustrating Step ST32, the cylindrical body 10 is disposed in the liquid-retaining receptacle 51 in which the liquid 66 containing the filtration targets 61 is retained. In Step ST32, the cylindrical body 10 is immersed in the liquid 66, which in turn passes through the filtration part 20 to flow into the cylindrical body 10. In this stage, the filtration targets 61 are caught in the filtration part 20. Consequently, the liquid 66 without the filtration targets 61 contained therein flows into the cylindrical body 10. In Embodiment 4, dead cells and/or dirt may pass through the filtration part 20 to flow into the cylindrical body 10.

The inflow of the liquid 66 into the cylindrical body 10 in Step ST32 is due to atmospheric pressure. No extra pressure is exerted on the liquid 66 while it flows into the cylindrical body 10, and damage to the filtration targets 61 may be reduced accordingly.

Embodiment 4 involves immersing the cylindrical body 10 in the liquid 66. The liquid permeability of the through-holes 21 of the filtration part 20 may be increased accordingly.

Referring to FIG. 27 and FIG. 28C illustrating Step ST33, the liquid 66 in the cylindrical body 10 is collected. In Step ST33, a collection tool 74 is used to collect the liquid 66 held in the cylindrical body 10. The collection tool 74 is, for example, a pipette or a syringe. Alternatively, the collection tool 74 may be a hollow tube connected to a pump.

The liquid 66 in the cylindrical body 10 is collected by suction through the use of the collection tool 74.

The tip of the collection tool 74 in Embodiment 4 is placed in such a manner that a tip thereof is within the reservoir part 30 in the lower portion of the cylindrical body 10. The filtration targets 61 are less affected by the suction force generated to suck the liquid 66 into the collection tool 74, and damage to the filtration targets 61 may be reduced accordingly.

Referring to FIG. 28D, the liquid 66 in the cylindrical body 10 is continuously collected through the use of the collection tool 74, and in due course of time, the surface of the liquid 66 in the liquid-retaining receptacle 51 comes down to a lower end 23 of the filtration part 20, that is, to the opening of the reservoir part 30, where the liquid 66 does not flow anymore into the cylindrical body 10. The filtration is then ended.

In Embodiment 4, adjusting the position of the lower end 23 of the filtration part 20 enables control of the collectable volume of the liquid 66.

The filtration device 1D and the filtration method according to Embodiment 4 produce the following effects.

According to the filtration method implemented with the filtration device 1D, filtration is carried out while the cylindrical body 10 is in contact with the liquid 66 containing the filtration targets 61 and retained in the liquid-retaining receptacle 51. This contributes to the enhanced filtration efficiency. Specifically, the depositing of the filtration targets 61 on the filtration part 20 may be minimized, and the liquid 66 containing the filtration targets 61 and retained in the liquid-retaining receptacle 51 may become concentrated.

Embodiment 4 describes that the filtration device 1D includes the reservoir part 30. In some embodiments, the reservoir part 30 of the filtration device 1D is optional. It is required that the filtration device 1D include: the cylindrical body 10 having two ends and having the opening 13 at first end and the end wall 12 at the second end; and the filtration part 20 provided on the circumferential portion 11 of the cylindrical body 10 and having the through-holes 21. This configuration suffices to produce the aforementioned effects; that is, the depositing of the filtration targets 61 on the filtration part 20 may be minimized, and the liquid 66 containing the filtration targets 61 and retained in the liquid-retaining receptacle 51 may become concentrated.

### Embodiment 5

The following describes a filtration device according to Embodiment 5 of the present invention with a focus on differences between Embodiment 4 and Embodiment 5. Each component described in Embodiment 4 and the corresponding (identical or similar) component that will be described in Embodiment 5 are denoted by the same reference sign. Description that holds true for both Embodiments 4 and 5 will be omitted.

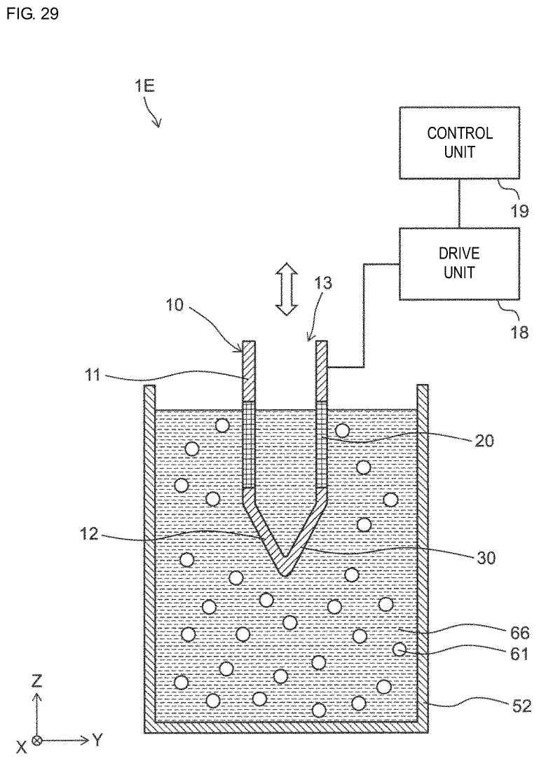

FIG. 29 is a schematic sectional view of an example of a filtration device 1E according to Embodiment 5 of the present invention. As illustrated in FIG. 29, Embodiment 5 differs from Embodiment 4 in that the filtration device 1E includes a constituent component capable of driving the cylindrical body 10 in a manner so as to cause the cylindrical body 10 to ascend or descend (in the Z direction).

Specifically, the filtration device 1E includes: the cylindrical body 10; the filtration part 20 on the circumferential portion 11 of the cylindrical body 10; the reservoir part 30 below the filtration part 20; and a liquid-retaining receptacle 52, in which the liquid 66 containing the filtration targets 61 is retained. The filtration device 1E also includes a drive unit 18 and a control unit 19 to cause the cylindrical body 10 to ascend or descend. The drive unit 18 is connected to the cylindrical body 10, and the control unit 19 controls the drive unit 18.

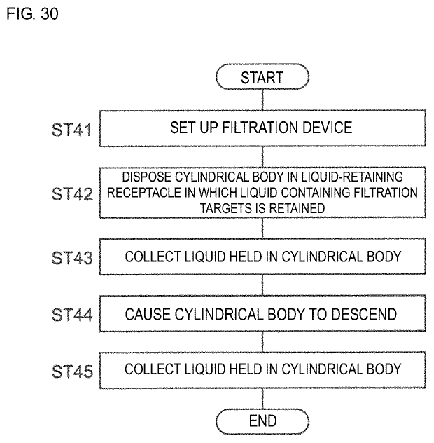

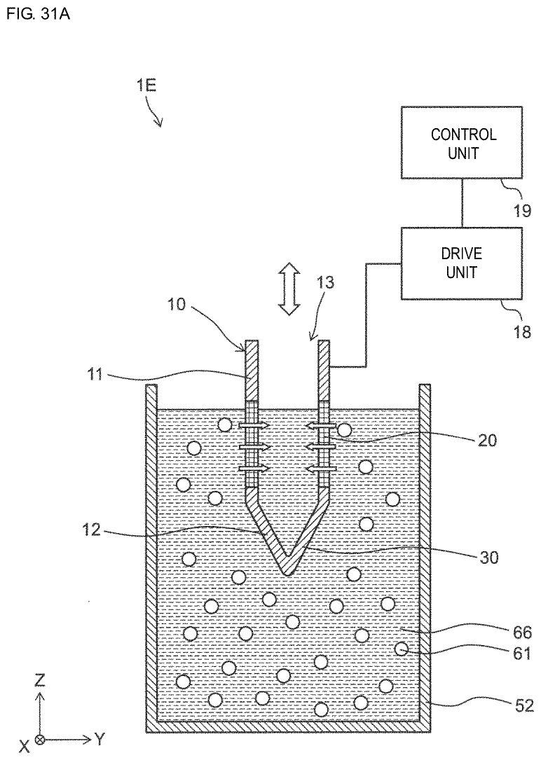

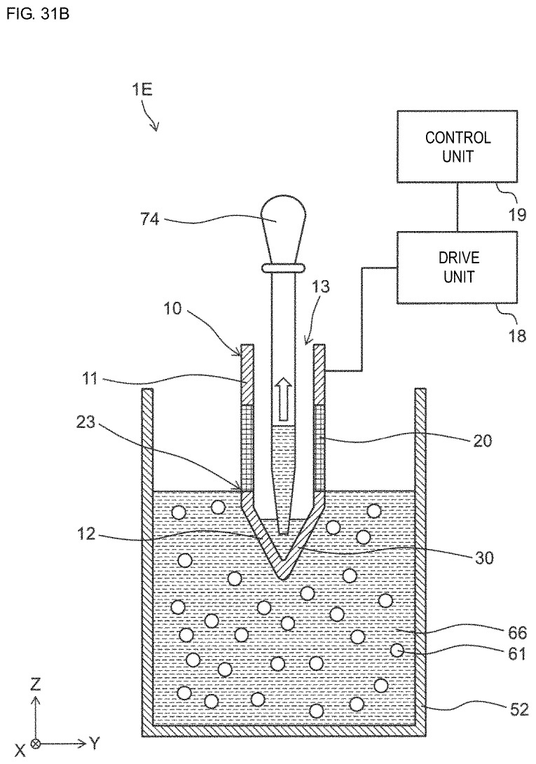

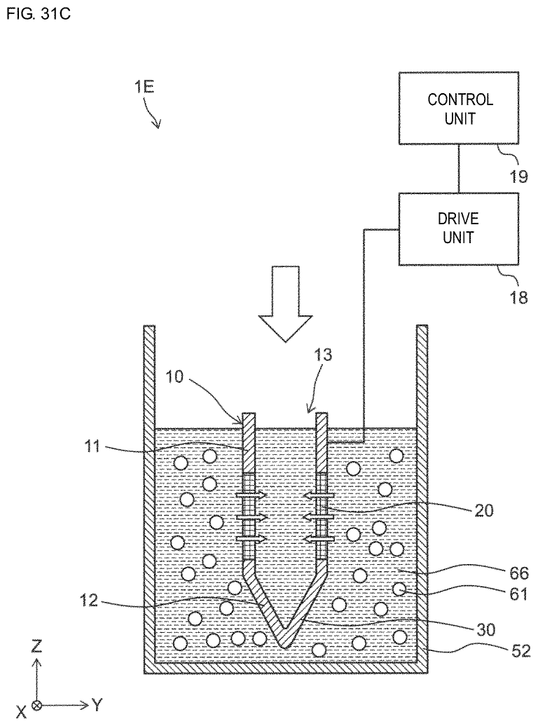

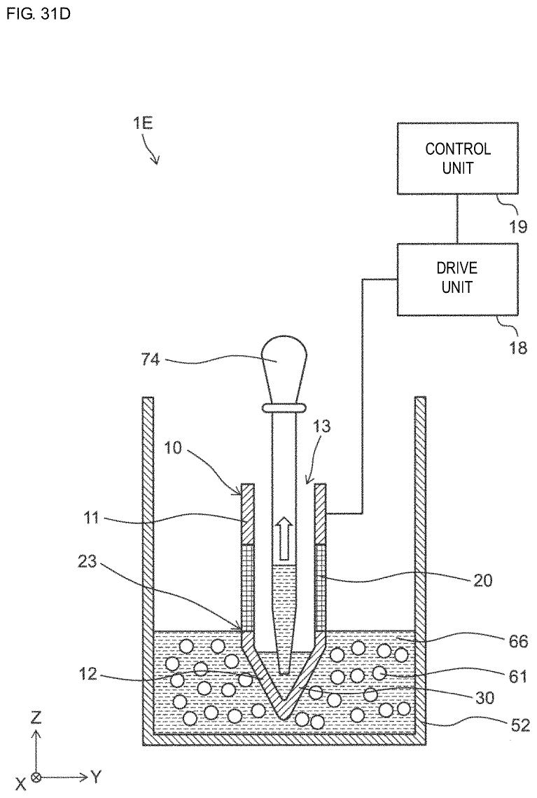

An example of a filtration method according to Embodiment 5 will be described below with reference to FIGS. 30 and 31A to 31D. FIG. 30 is a flowchart of an example of the filtration method according to Embodiment 5 of the present invention. FIGS. 31A to 31D illustrate steps that may be included in the filtration method according to Embodiment 5 of the present invention.

Referring to FIG. 30, the filtration device 1E is set up in Step ST41 (see FIG. 29). In Embodiment 5, the liquid 66 in the liquid-retaining receptacle 52 is a cell suspension, and the filtration targets 61 are cells. The liquid-retaining receptacle 52 is a beaker, a test tube, a tank, or any other receptacle in which the liquid 66 may be retained.

Referring to FIG. 30 and FIG. 31A illustrating Step ST42, the cylindrical body 10 is disposed in the liquid-retaining receptacle 52 in which the liquid 66 containing the filtration targets 61 is retained. In Step ST42, the cylindrical body 10 is immersed in the liquid 66, which in turn passes through the filtration part 20 to flow into the cylindrical body 10. In this stage, the filtration targets 61 are caught in the filtration part 20. Consequently, the filtration targets 61 do not get into the cylindrical body 10; that is, the liquid 66 without the filtration targets 61 contained therein flows into the cylindrical body 10.

Referring to FIG. 30 and FIG. 31B illustrating Step ST43, the liquid 66 in the cylindrical body 10 is collected. In Step ST43, the collection tool 74 is used to collect the liquid 66 held in the cylindrical body 10. In Embodiment 5, the tip of the collection tool 74 is placed in the reservoir part 30, and the liquid 66 in the cylindrical body 10 is collected by suction through the tip of the collection tool 74. The liquid 66 in the cylindrical body 10 may be continuously collected through the use of the collection tool 74 until the surface of the liquid 66 in the liquid-retaining receptacle 52 comes down to the lower end 23 of the filtration part 20, where the liquid 66 does not flow anymore into the cylindrical body 10 through the filtration part 20.

Referring to FIG. 30 and FIG. 31C illustrating Step ST44, the drive unit 18 causes the cylindrical body 10 to descend. In Embodiment 5, the drive unit 18 is controlled by the control unit 19. For example, the control unit 19 obtains, from a detection unit, information about the position of the surface of the liquid 66 retained in the liquid-retaining receptacle 52 and information about the position of the cylindrical body 10. On the basis of the information obtained, the control unit 19 controls the drive unit 18, which in turn causes the cylindrical body 10 to descend.

Once the cylindrical body 10 descends, the inflow of the liquid 66 resumes; that is, the liquid 66 retained in the liquid-retaining receptacle 52 passes through the filtration part 20 to flow into the cylindrical body 10.

Referring to FIG. 30 and FIG. 31D illustrating Step ST45, the liquid 66 in the cylindrical body 10 is collected. As in Step ST43, the collection tool 74 is used in Step ST45 to collect the liquid 66 held in the cylindrical body 10.

The liquid 66 in the cylindrical body 10 is continuously collected through the use of the collection tool 74, and in due course of time, the surface of the liquid 66 in the liquid-retaining receptacle 51 comes down to the lower end 23 of the filtration part 20, that is, to the opening of the reservoir part 30, where the liquid 66 does not flow anymore into the cylindrical body 10. The filtration is then ended.

As in Embodiment 4, adjusting the position of the lower end 23 of the filtration part 20 in Embodiment 5 enables control of the collectable volume of the liquid 66.

The filtration device 1E and the filtration method according to Embodiment 5 produce the following effects.

According to the filtration method implemented with the filtration device 1E, filtration is carried out while the cylindrical body 10 is in contact with the liquid 66 containing the filtration targets 61 and retained in the liquid-retaining receptacle 52. The constituent components for causing the cylindrical body 10 to ascend or descend are included. This contributes to the enhanced filtration efficiency. Specifically, the depositing of the filtration targets 61 on the filtration part 20 may be minimized, and the liquid 66 containing the filtration targets 61 and retained in the liquid-retaining receptacle 52 may become concentrated. Driving the cylindrical body 10 in a manner so as to cause the cylindrical body 10 to ascend or descend enables control of the volume of the liquid 66 left in the liquid-retaining receptacle 52.

In other words, the volume of liquid remaining in the liquid-retaining receptacle 52 may be controlled in a manner so as to adjust the liquid thickness.

Embodiment 5 describes that the drive unit 18 causes the cylindrical body 10 to descend. In some embodiments, the drive unit 18 may cause the cylindrical body 10 to ascend. For example, when the opening 13 of the cylindrical body 10 is located at a level below the surface of the liquid 66 in the liquid-retaining receptacle 52, the drive unit 18 may cause the cylindrical body 10 to ascend.

Embodiment 5 describes that Step ST44, namely, the step of causing the cylindrical body 10 to move is independent of Steps ST43 and ST45, namely, the steps of collecting the liquid 66. In some embodiments, Steps ST43 to ST45 may be performed at the same time.

For example, the filtration method according to Embodiment 5 may be modified as follows: the liquid 66 in the cylindrical body 10 is collected through the use of the collection tool 74 while the drive unit 18 causes the cylindrical body 10 to descend. This is conducive to a short-time filtration, which contributes to the further enhanced filtration efficiency.

Embodiment 5 describes that the filtration device 1E includes the drive unit 18 and the control unit 19 to cause the cylindrical body 10 to ascend or descend. In some embodiments, the filtration device 1E may include any other constituent component capable of causing the cylindrical body 10 to move in the height direction (i.e., in the Z direction).

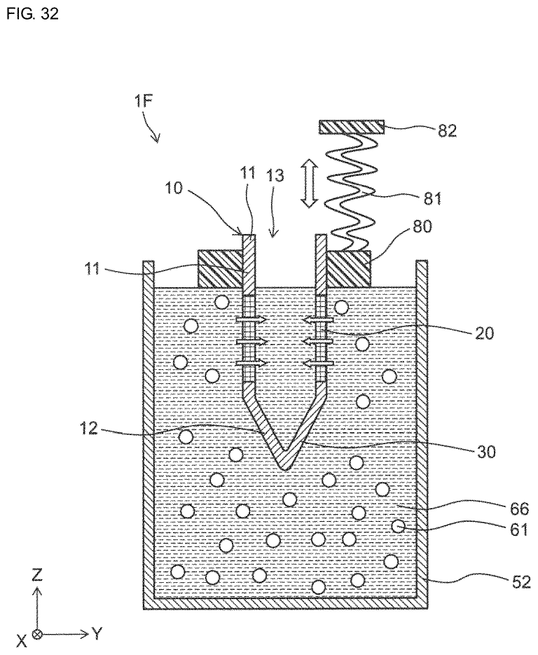

FIG. 32 is a schematic sectional view of an example of a filtration device 1F according to a modification of Embodiment 5 of the present invention. As illustrated in FIG. 32, the filtration device 1F includes constituent components for causing the cylindrical body 10 to move in the height direction, or more specifically, a float 80, a connection line 81, and a fixed part 82. The float 80 is connected to the cylindrical body 10. The connection line 81 is connected to the float 80. The fixed part 82 is connected to the connection line 81. The other constituent components of the filtration device 1F are identical to the respective constituent components of the filtration device 1E.

The float 80 is connected to the circumferential portion 11 of the cylindrical body 10. Specifically, the float 80 is disposed above the filtration part 20. The float 80 floats in the liquid 66 in a manner so as to retain the cylindrical body 10. That is, the float 80, together with the cylindrical body 10, floats in the liquid 66. The cylindrical body 10 is retained on or immediately below the surface of the liquid 66 accordingly.

The connection line 81 is connected to the float 80 and to the fixed part 82. Specifically, one end of the connection line 81 is connected to the float 80, and the other end of the connection line 81 is connected to the fixed part 82. The connection line 81 of the filtration device 1F is length adjustable to allow adjustment of the volume of the liquid 66 remaining in the liquid-retaining receptacle 52.

The connection line 81 is slack while the cylindrical body 10 is retained by the float 80 and floats in the liquid 66. As the liquid 66 in the cylindrical body 10 is continuously collected through the use of the collection tool 74, the surface of the liquid 66 in the liquid-retaining receptacle 52 comes down. As the surface of the liquid comes down, the connection line 81 is stretched downward. When the connection line 81 is stretched to its full length, the cylindrical body 10 stops descending. That is, the cylindrical body 10 is retained by the connection line 81 stretched to its full length.

The fixed part 82 is connected to the connection line 81. The fixed part 82 is fixed to a part other than the cylindrical body 10 and the float 80. For example, the fixed part 82 may be fixed to the liquid-retaining receptacle 52.

The filtration device 1F is configured as follows: the liquid 66 in the cylindrical body 10 is collected through the use of the collection tool 74 in the state in which the float 80 retaining the cylindrical body 10 floats in the liquid 66. As the liquid 66 in the cylindrical body 10 is collected through the use of the collection tool 74, the surface of the liquid 66 in the liquid-retaining receptacle 52 comes down. The float 80 floats in the liquid 66 and retains the cylindrical body 10 accordingly. As the surface of the liquid 66 comes down, the cylindrical body 10 descends.

The fixed part 82 is fixed to, for example, the liquid-retaining receptacle 52. One end of the connection line 81 is connected to the float 80, and the other end of the connection line 81 is connected to the fixed part 82. As the float 80 descends, the connection line 81 is stretched downward. When the connection line 81 is stretched to its full length, the cylindrical body 10 is retained by the connection line 81 and stops descending.

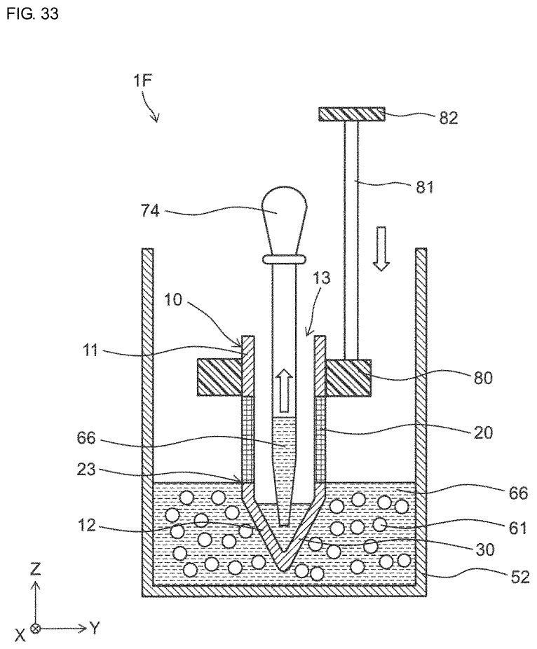

FIG. 33 is a schematic sectional view of the filtration device 1F according to the modification of Embodiment 5 of the present invention, illustrating an action that may be performed by the filtration device 1F. As illustrated in FIG. 33, the cylindrical body 10 is retained by the connection line 81 stretched to its full length. Consequently, the cylindrical body 10 does not descend anymore while the surface of the liquid 66 in the liquid-retaining receptacle 52 continues to come down. In this state, the liquid 66 in the cylindrical body 10 is collected through the use of the collection tool 74.

The liquid 66 is continuously collected through the use of the collection tool 74, and in due course of time, the surface of the liquid 66 in the liquid-retaining receptacle 52 comes down below the lower end 23 of the filtration part 20, where the liquid 66 does not flow anymore into the cylindrical body 10 through the filtration part 20. The volume of the liquid 66 in the liquid-retaining receptacle 52 may be adjusted accordingly.

As mentioned above, the float 80 and the connection line 81 of the filtration device 1F enable adjustment of the volume of the liquid 66 remaining in the liquid-retaining receptacle 52. Specifically, the connection line 81 is length adjustable to allow adjustment of the position of the cylindrical body 10 in the height direction (i.e., in the Z direction). The volume of the liquid 66 remaining in the liquid-retaining receptacle 52 may be adjusted accordingly.

### Embodiment 6

The following describes a filtration device according to Embodiment 6 of the present invention with a focus on differences between Embodiment 4 and Embodiment 6. Each component described in Embodiment 4 and the corresponding (identical or similar) component that will be described in Embodiment 6 are denoted by the same reference sign. Description that holds true for both Embodiments 4 and 6 will be omitted.

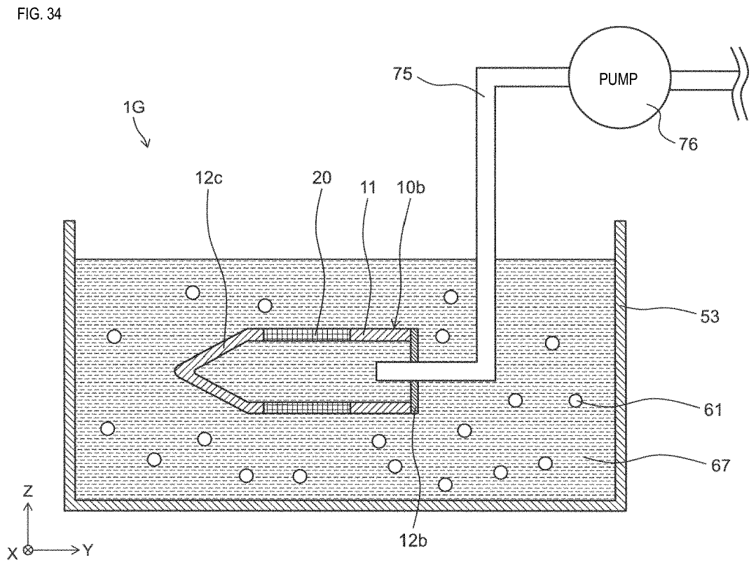

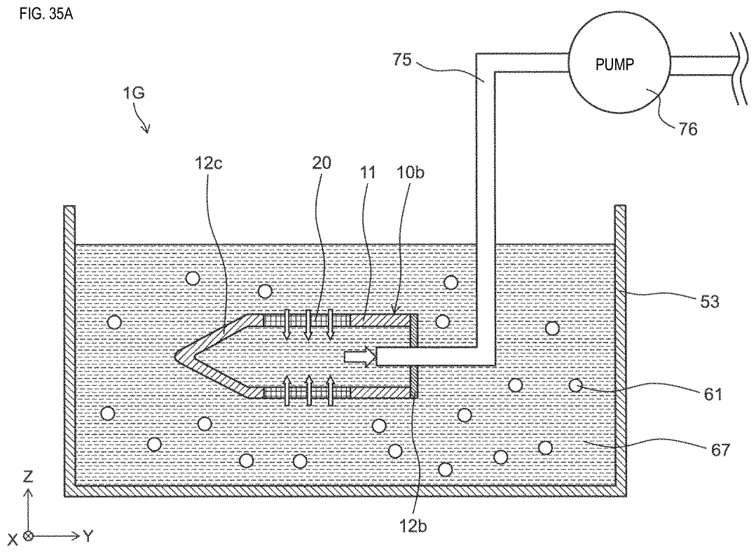

FIG. 34 is a schematic sectional view of an example of a filtration device 1G according to Embodiment 6 of the present invention. As illustrated in FIG. 34, Embodiment 6 differs from Embodiment 4 in that filtration is carried out with the cylindrical body 10 horizontally oriented (in the X and Y directions).

The filtration device 1G includes a cylindrical body 10b and the filtration part 20. The cylindrical body 10b has two ends. The cylindrical body 10b has a first end wall 12b and a second end wall 12c. A first end of the cylindrical body 10b is closed with the first end wall 12b, and the second end of the cylindrical body 10b is closed with the second end wall 12c. The filtration part 20 is provided on the circumferential portion 11 of the cylindrical body 10b and has the through-holes 21. The filtration device 1G also includes a hollow tube 75 and a pump 76. The hollow tube 75 extends through the first end wall 12b. The pump 76 is connected to the hollow tube 75. The filtration device 1G includes a liquid-retaining receptacle 53, in which a liquid 67 is retained. The liquid 67 contains the filtration targets 61.

In Embodiment 6, the liquid 67 is a cell suspension, and the filtration targets 61 are cells.

The first end wall 12b has a through-hole through which the hollow tube 75 passes. A tip of the hollow tube 75 is inserted into the cylindrical body 10b through the through-hole in the first end wall 12b.

The second end wall 12c in Embodiment 6 is recessed in the longitudinal direction of the cylindrical body 10b (i.e., in the Y direction). The filtration part 20 extends all around the circumferential portion 11 of the cylindrical body 10b.

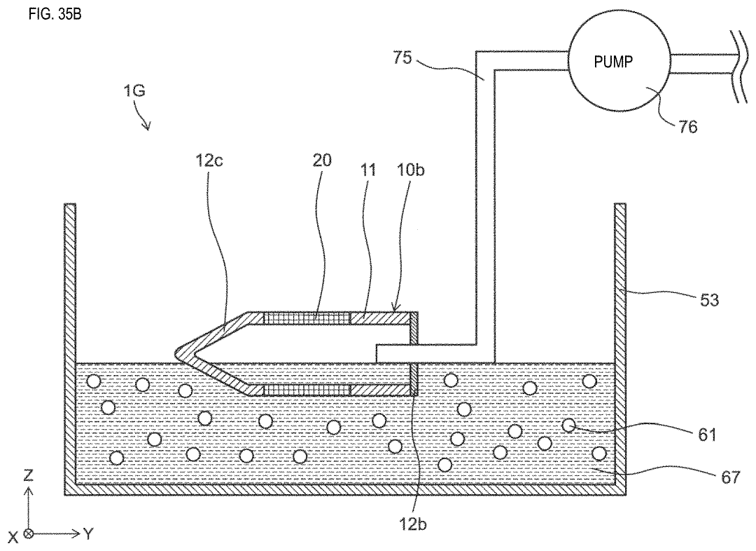

Actions that may be performed by the filtration device 1G (i.e., an example of a filtration method) will be described below with reference to FIGS. 35A and 35B. FIGS. 35A and 35B illustrate actions that may be performed by the filtration device 1G according to Embodiment 6 of the present invention.

Referring to FIG. 35A, the cylindrical body 10b is laid horizontally (i.e., in the X and Y directions) in the liquid-retaining receptacle 53. Consequently, the cylindrical body 10b is immersed in the liquid 67 containing the filtration targets 61. The liquid 67 passes through the filtration part 20 to flow into the cylindrical body 10b whereas the filtration targets 61 are caught in the filtration part 20.

The horizontal orientation of the cylindrical body 10b is conducive to promoting inflow of the liquid 67 into the cylindrical body 10b. The liquid 67 in the cylindrical body 10b may be collected under weaker suction pressure than if the cylindrical body 10b is vertically oriented.

The hollow tube 75 and the pump 76 are used to collect the liquid 67 held in the cylindrical body 10b. Specifically, the pump 76 performs, through the hollow tube 75, suctioning of the liquid 67 held in the cylindrical body 10b. The liquid 67 in the liquid-retaining receptacle 53 passes through the filtration part 20 to flow into the cylindrical body 10b and is then collected through the use of the pump 76 and the hollow tube 75.

Referring to FIG. 35B, the liquid 67 in the cylindrical body 10b is continuously collected until the surface of the liquid 67 in the liquid-retaining receptacle 53 comes down to a lower end of the hollow tube 75.

The filtration device 1G and the filtration method according to Embodiment 6 produce the following effects.

According to the filtration method implemented with the filtration device 1G the liquid 67 containing the filtration targets 61 is filtered with the cylindrical body 10b horizontally oriented (in the X and Y directions) in the liquid-retaining receptacle 53. This contributes to the enhanced filtration efficiency. Specifically, the horizontal orientation of the cylindrical body 10b is conducive to promoting inflow of the liquid 67 into the cylindrical body 10b through the filtration part 20. According to the filtration method implemented with the filtration device 1G, the liquid in the cylindrical body may be collected under weaker suction pressure than if the cylindrical body is vertically oriented (in the Z direction). Thus, the cells are more protected from damage caused by the pressure. This helps keep the cells active.

Embodiment 6 describes that the second end wall 12c is recessed in the longitudinal direction of the cylindrical body 10b (i.e., in the Y direction). In some embodiments, the second end wall 12c may have the shape of a flat plate.

Embodiment 6 describes that the filtration device 1G includes the hollow tube 75 and the pump 76 that are used to collect the liquid 67 held in the cylindrical body 10b. In some embodiments, the pump 76 may be omitted from the filtration device 1G; and the liquid 67 may be collected though the hollow tube 75 disposed at a level below the cylindrical body 10b.

Embodiment 6 describes that the filtration part 20 extends all around the circumferential portion 11 of the cylindrical body 10b. In some embodiments, the filtration part 20 may extend at least partially around the circumferential portion 11 of the cylindrical body 10b.

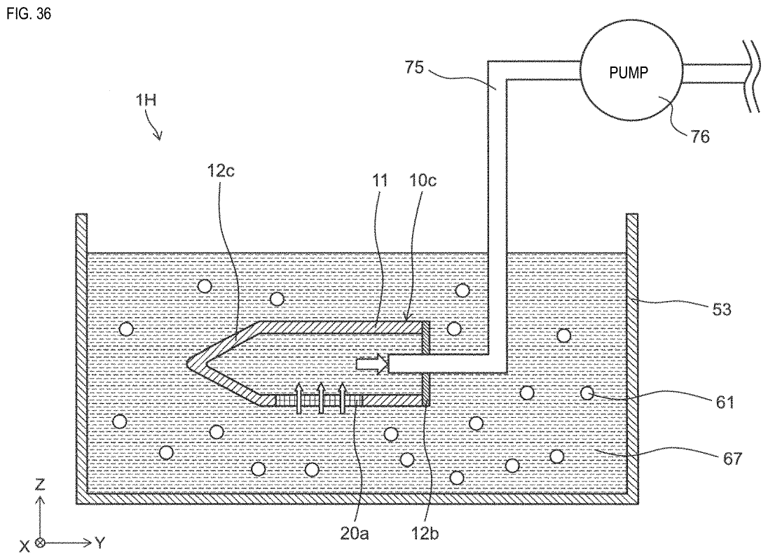

FIG. 36 is a schematic sectional view of a filtration device 1H according to a modification of Embodiment 6 of the present invention. As illustrated in FIG. 36, the filtration device 1H may include a filtration part 20a, which extends halfway or less around the circumferential portion 11.

According to a filtration method implemented with the filtration device 1H, a cylindrical body 10c is laid horizontally (in the X and Y directions) in such a manner that a region being part of the circumferential portion 11 of the cylindrical body 10c and overlaid with the filtration part 20a is located at a level below the remainder of the circumferential portion 11 that is not overlaid with the filtration part 20a. Thus, the through-holes 21 of the filtration part 20a are less likely to be blocked by the filtration targets 61 that have settled out. That is, the filtration device 1H minimizes clogging of the filtration part 20a, and damage to the filtration targets 61 may be reduced accordingly.

### Example 1

In Example 1, a cell suspension was subjected to cross-flow filtration carried out by the filtration device 1A according to Embodiment 1 and was then collected from the reservoir part 30. Subsequently, the cell-suspension (i.e., liquid) collection rate and the cell collection rate were determined. The conditions of the cell suspension in Example 1 are presented in Table 1. An image analysis cell counter device (Countess II FL Automated Cell Counter manufacture by Thermo Fisher) was used to determine the cell concentration. The trypan blue exclusion method was used to test cell vitality.

TABLE 1

| | |
|---|---|
| Cells | HL-60 |
| Cell Size | 12 μm |
| Concentration of Introduced Cells | 2 × 10⁶ cells/ml |
| Volume of Introduced Liquid | 2 ml |

Specifications of the filtration device 1A in Example 1 are presented in Table 2.

TABLE 2

| | |
|---|---|
| Outside Diameter of Cylindrical body | 11 mm |
| Inside Diameter of Cylindrical body | 9 mm |
| Height of Cylindrical body | 47 mm |
| Outside Diameter of Filtration Part | 12 mm |
| Thickness of Filtration Part | 2 μm |
| Shape of Through-Holes | square |
| Arrangement of Through-Holes | square grid array |
| Size of Through-Holes | each side 6 μm in length |
| Interval between Through-Holes | 8.5 μm |
| Aperture Ratio | 50% |
| Capacity of Reservoir Part | 1 ml |

In Example 1, eight experiments were carried out under the same conditions. Each experiment was carried out as will be described hereinafter. Two milliliters of cell suspension mentioned in Table 1 were introduced into the filtration device 1A and were allowed to stand for two minutes, at the end of which the liquid 60 was not drained anymore from the filtration part 20. Subsequently, the cell suspension in the reservoir part 30 was collected through the use of a pipette. Then, the volume of the collected cell suspension and the number of collected cells were determined by measurement, and the cell-suspension collection rate relative to the liquid collection target volume (one milliliter) and the cell collection rate were determined by calculation. The liquid volume was determined by reading the graduation mark on the pipette, and the cell concentration was determined by using the cell counter mentioned above. Table 3 shows the cell-suspension collection rate and the cell collection rate that were determined by calculation. The cell-suspension collection rate relative to the liquid collection target volume (one milliliter) in Table 3 was obtained by dividing the volume of the collected cell suspension by one milliliter and by multiplying the quotient by 100. The cell collection rate was obtained by dividing the number of living cells contained in the collected cell suspension by 4×10⁶ and by multiplying the quotient by 100.

TABLE 3

| | Example 1 | | | | | | | |
|---|---|---|---|---|---|---|---|---|
| | 1st | 2nd | 3rd | 4th | 5th | 6th | 7th | 8th |
| | EXPT | EXPT | EXPT | EXPT | EXPT | EXPT | EXPT | EXPT |
| Volume of | 1 ml | 0.8 ml | 0.9 ml | 1.1 ml | 1.2 ml | 0.7 ml | 1 ml | 1.1 ml |
| Collected | | | | | | | | |
| Cell | | | | | | | | |
| Suspension | | | | | | | | |
| Cell- | 100% | 80% | 90% | 110% | 120% | 70% | 100% | 110% |
| Suspension | | | | | | | | |
| Collection | | | | | | | | |
| Rate | | | | | | | | |
| Relative to | | | | | | | | |
| Liquid | | | | | | | | |
| Collection | | | | | | | | |
| Target | | | | | | | | |
| Volume | | | | | | | | |
| (1 ml) | | | | | | | | |
| Cell | 80% | 96% | 83% | 86% | 88% | 90% | 99% | 78% |
| Collection | | | | | | | | |
| Rate | | | | | | | | |

Table 3 indicates that the filtration device 1A attained high cell collection rates; that is, cells were easily collected. The obtained cell-suspension collection rates relative to the liquid collection target volume (one milliliter) were also high. This indicates that the cell suspension in the reservoir part 30 was adequately collected; that is, a desired volume of liquid was obtained. The cells collected in Example 1 were still active. Example 1 thus proved to be a low-damage procedure for cells.

### Example 2

In Example 2, a cell suspension was filtered by the filtration device 1C according to Embodiment 2 with the cylindrical body 10 immersed in the first liquid (PBS). The cell suspension was then allowed to stand for two minutes. Subsequently, two milliliters of PBS were injected to wash cells. The cell suspension in the reservoir part 30 was then collected through the use of a pipette, and the cell collection rate was determined. The conditions of the cell suspension in Example 2 are presented in Table 4. Specifications of the filtration device 1C in Example 2 are identical to the specifications of the filtration device 1A in Example 1 (see Table 2).

TABLE 4

| | |
|---|---|
| Cells | HL-60 |
| Cell Size | 12 μm |
| Concentration of Introduced Cells | 2.05 × 10⁶ cells/ml |
| Volume of Introduced Liquid | 2 ml |

In Reference Example 1, filtration of a cell suspension in the atmosphere was followed by washing of cells in the atmosphere, and the cell suspension was then collected to determine the cell collection rate. Reference Example 1 involved the use of a cylindrical body, a filtration part on a circumferential portion of the cylindrical body, and a reservoir part below the filtration part. Reference Example 1 differs from Example 2 in that the first liquid was not used. That is, Reference Example 1 differs from Embodiment 2 in that filtration and washing were carried out in the atmosphere without the cylindrical body being immersed in liquid.

Table 5 shows the cell collection rate determined in Example 2.

TABLE 5

| | Experiments | | | |
|---|---|---|---|---|
| | 1st | 2nd | 3rd | 4th |
| | EXPT | EXPT | EXPT | EXPT |
| Cell Collection Rate (%) | 85 | 82 | 88 | 73 |

Subsequent to the procedure mentioned above, the cylindrical body 10 was immersed again in the PBS retained in the liquid-retaining receptacle 50, and two milliliters of PBS were then injected through the opening 13. The cylindrical body 10 was lifted out of the liquid-retaining receptacle 50 to collect the cell suspension. Table 6 shows the results. The sum of the cell collection rate in Table 5 and the cell collection rate in Table 6 is presented in the lowermost row of Table 6.

TABLE 6

| | Experiments | | | |
|---|---|---|---|---|
| | 1st | 2nd | 3rd | 4th |
| | EXPT | EXPT | EXPT | EXPT |
| Number of Collected Cells (×10⁶) | 0.3 | 0.47 | 0.28 | 0.38 |
| Cell Collection Rate (%) | 7 | 11 | 7 | 9 |
| Sum of Cell Collection Rates (%) | 92 | 93 | 95 | 81 |

Table 7 shows the cell collection rate determined in Reference Example 1.

TABLE 7

| | Experiments | | | |
|---|---|---|---|---|
| | 1st | 2nd | 3rd | 4th |
| | EXPT | EXPT | EXPT | EXPT |
| Cells | HL-60 | | | |
| Cell Size | 12 μm | | | |
| Number of Introduced Cells (×10⁶) | 4.1 | 4.1 | 4.1 | 4.1 |
| Number of Collected Cells (×10⁶) | 1.77 | 2.38 | 2.02 | 2.43 |
| Cell Collection Rate (%) | 43 | 58 | 49.2 | 59.2 |

As presented in the lowermost row of Table 6, the cell collection rates in Example 2 were as high as 92%, 93%, 95%, and 81%. Meanwhile, the cell collection rates in Reference Example 1 were 43%, 58%, 49.2%, and 59.2%, which are shown in Table 7. This has proven that Example 2 can offer an improvement, in terms of cell collection rate, over Reference Example 1.

In Reference Example 1, filtration in the atmosphere was followed by washing of cells in the atmosphere. Specifically, cells were washed with two milliliters of washing solution (PBS) introduced into the cylindrical body 10 in the atmosphere through the opening of the cylindrical body. The introduction of the washing solution caused stirring of the cells in the cylindrical body, and as a result, some of the cells became deposited on the filtration part. The washing solution was then drained from the filtration part 20, which presumably got clogged with the cells pressed into the through-holes of the filtration part. This is probably the reason that the collection rates in Reference Example 1 were lower than the collection rates in Example 2. When the washing solution is used in higher amounts or introduced at a higher speed, clogging is more likely to occur and can accordingly cause a reduction in collection rate.

In Example 2, the cylindrical body 10 was kept immersed in liquid during filtration and washing. Consequently, the number of cells pressed against the filtration part was reduced. Specifically, the solution in Example 2 and the washing solution diffused by passing through the filtration part 20 during the immersion filtration and the immersion washing. The speed of the liquid passing through the filtration part 20 in Example 1 was therefore not as fast as the speed of the liquid passing through the filtration part in Reference Example 1. Consequently, the number of cells pressed against the filtration part was reduced, and the occurrence of clogging was reduced accordingly. This is probably the reason that the collection rates in Example 2 were higher than the collection rates in Reference Example 2.

While the present invention has been thoroughly described so far by way of preferred embodiments with reference to the accompanying drawings, variations and modifications will be apparent to those skilled in the art. It should be understood that the variations and modifications made without departing from the scope hereinafter claimed are also embraced by the present invention.

### INDUSTRIAL APPLICABILITY

The filtration device according to the present invention is useful in industrial fields involving commonly-used filtration procedures. Cells may remain active during filtration carried out by the filtration device, which is therefore particularly useful in, for example, drug efficacy research and production of drugs for regenerative medicine.

### REFERENCE SIGNS LIST

1A, 1AA, 1AB, 1AC, 1AD, 1AE, 1AF, 1AG, 1BA, 1BB, 1BC, 1BD, 1C, 1D, 1E, 1F, 1G; 1H filtration device
10, 10b, 10ba, 10bb, 10bc cylindrical body
11, 11ba, 11bc circumferential portion
11aa flange portion
12, 12ba end wall
12b first end wall
12c second end wall
13, 13bc opening
14 frame member
15 opening
16 inner surface
17 outer surface
18 drive unit
19 control unit
20, 20a, 20ac, 20ad, 20ae filtration part
21 through-hole
22 filter substrate
23 lower end
30, 30aa, 30ab, 30ba, 30bb, 30bc, 30bd reservoir part
31 connection portion
32 lowermost end portion
33, 33aa, 33ba inner wall
34, 34ba outer wall
35 inclined portion
36 protruding portion
37 valve
40 liquid-retaining receptacle
41 bottom portion
42 side wall
43 opening
50, 51, 52, 53 liquid-retaining receptacle
60 liquid
61 filtration target
62, 63, 64, 65, 66, 67 liquid
70 collection tool
71, 72, 73 pipette
74 collection tool
75 hollow tube
76 pump
80 float
81 connection line
82 fixed part
90 tab part
91 lid
100A, 100B, 100C filtration system
101, 101a, 101b liquid-retaining receptacle
102, 102a channel
103 valve
104, 104a waste liquid receptacle
105 waste liquid channel
106 switching valve
107 sample receptacle
108, 108a collection receptacle
110 waste liquid
111 liquid
120 feed opening
121 receptacle
122 channel
123a first waste liquid outlet
123b second waste liquid outlet
124 channel
125 channel
126 collection port
127 channel
128a, 128b, 128c, 128d valve
129 filter

1. A filtration device, comprising:
a cylindrical body having:
a first open end;
a second closed end opposite the first open end; and
a plurality of frame members that define openings between an inside and an outside of the cylindrical body; and
a cylindrical filter having through holes, the cylindrical filter being attached to the plurality of frame members so as to wrap around a circumferential portion of the cylindrical body.

2. The filtration device according to claim 1, wherein the cylindrical filter extends all around the circumferential portion of the cylindrical body.

3. The filtration device according to claim 1, wherein the cylindrical filter extends halfway or less around the circumferential portion of the cylindrical body.

4. The filtration device according to claim 1, wherein the second closed end is configured as a reservoir part.

5. The filtration device according to claim 4, wherein, when cross sections of the reservoir part are taken in directions orthogonal to a direction of connection between the first open end and the second closed end of the cylindrical body, an opening cross-sectional area of a portion of the reservoir part closer to the second closed end of the cylindrical body is smaller than an opening cross-sectional area of a portion of the reservoir part closer to the cylindrical filter.

6. The filtration device according to claim 5, wherein the reservoir part has an inner wall including an inclined portion inclined toward the second closed end of the cylindrical body.

7. The filtration device according to claim 6, wherein the inclined portion is inclined toward a center of the cylindrical body.

8. The filtration device according to claim 4, wherein the reservoir part has an outer wall including a protruding portion protruding toward the second closed end of the cylindrical body.

9. The filtration device according to claim 8, wherein the protruding portion has a side face inclined toward a center of the cylindrical body.

10. The filtration device according to claim 1, further comprising a liquid-retaining receptacle close to the second closed end of the cylindrical body.

11. The filtration device according to claim 1, wherein the cylindrical body is made of a transparent resin.

12. The filtration device according to claim 1, wherein the cylindrical filter is mainly comprised of a metal and/or a metal oxide.

13. The filtration device according to claim 1, wherein a surface of the cylindrical filter has a surface roughness smaller than a size of a filtration target.

14. The filtration device according to claim 13, wherein the surface roughness is smaller than half the size of the filtration target.

15. The filtration device according to claim 1, wherein at least a part of the circumferential portion of the cylindrical body between the first open end and the cylindrical filter is not fully surrounded by the cylindrical filter.

16. The filtration device according to claim 1, further comprising a flange portion extending radially from the first open end of the cylindrical body.

17. A filtration method comprising:
setting up a filtration device including a cylindrical body, a filtration part, and a reservoir part, the cylindrical body having a first open end, a second closed end, a plurality of frame members that define openings between an inside and an outside of the cylindrical body, the filtration part including a cylindrical filter having through holes attached to the plurality of frame members so as to wrap around a circumferential portion of the cylindrical body, the reservoir part being located at the second closed end of the cylindrical body and configured to store a filtration target and a liquid;
introducing a liquid containing a filtration target into the filtration device;
storing the filtration target and the liquid in the reservoir part;
draining the liquid from the filtration part, with the filtration target being caught in the filtration part; and
collecting the filtration target and the liquid from the reservoir part.

18. The filtration method according to claim 17, wherein the filtration device further includes a liquid-retaining receptacle closer to the second closed end of the cylindrical body than the first open end, and the method further comprises:
retaining the liquid drained from the filtration part in the liquid-retaining receptacle when draining the liquid from the filtration part.
